SEQUENCE LISTING (30 sequences)

SEQ ID NO: 1 - DNA, 31 bp, Homo sapiens
cagtcgacgagatggctcagtggttaagagc

SEQ ID NO: 2 - DNA, 32 bp, Homo sapiens
gatgcggccgcagccaagtgtgaatatacagc

SEQ ID NO: 3 - DNA, 32 bp, Homo sapiens; identical to SEQ ID NO: 2

SEQ ID NO: 4 - DNA, 28 bp, Homo sapiens
cagtcgacagggagcttgggacggatgc

SEQ ID NO: 5 - DNA, 23 bp, Homo sapiens
accgtaaagggcgtgtgccgcat

SEQ ID NO: 6 - DNA, 23 bp, Homo sapiens
aaacatgcggcacacgcccttta

SEQ ID NO: 7 - DNA, 23 bp, Homo sapiens
accgcatgtttgagaaactggaa

SEQ ID NO: 8 - DNA, 23 bp, Homo sapiens
aaacttccagtttctcaaacatg

SEQ ID NO: 9 - DNA, 23 bp, Homo sapiens
accgtacgatggtgagcgagtcc

SEQ ID NO: 10 - DNA, 23 bp, Homo sapiens
aaacggactcgctcaccatcgta

SEQ ID NO: 11 - DNA, 22 bp, Homo sapiens
ccaagaaggatgcacagagaaa

SEQ ID NO: 12 - DNA, 17 bp, Homo sapiens
gtaaagggcgtgtgccg

SEQ ID NO: 13 - DNA, 22 bp, Homo sapiens
gctctccagagaggtcatctaa

SEQ ID NO: 14 - DNA, 26 bp, Homo sapiens
acattaagtgcatgtttgagaaactg

SEQ ID NO: 15 - DNA, 20 bp, Homo sapiens
ttgtctcaggcagctctttc

SEQ ID NO: 16 - DNA, 20 bp, Homo sapiens
caagtacgatggtgagcgag

SEQ ID NO: 17 - DNA, 22 bp, Homo sapiens
catttgctttctccatcccaag

SEQ ID NO: 18 - DNA, 17 bp, Homo sapiens
gctacttcagccgcagt

SEQ ID NO: 19 - DNA, 20 bp, Homo sapiens
tgagcgaggaagcggaagag

SEQ ID NO: 20 - DNA, 20 bp, Homo sapiens
tcaggctgcgcaactgttgg

SEQ ID NO: 21 - DNA, 22 bp, Homo sapiens
tgccagtgagttgaccttaatc

SEQ ID NO: 22 - DNA, 21 bp, Homo sapiens
aagaagtgctggcgttctatc

SEQ ID NO: 23 - DNA, 34 bp, Homo sapiens
gcgaattcgccaccatggaatcaatcgaacaaca

SEQ ID NO: 24 - DNA, 29 bp, Homo sapiens
gctctagagtcagctacccagcaaacgca

SEQ ID NO: 25 - DNA, 24 bp, Homo sapiens
aaagcatctggcctacacccagaa

SEQ ID NO: 26 - DNA, 24 bp, Homo sapiens
accctcgtttagccgttcatgcta

SEQ ID NO: 27 - DNA, 21 bp, Homo sapiens
ccacgtgctctgtatgagatt

SEQ ID NO: 28 - DNA, 21 bp, Homo sapiens
atgctggcttatctgttcctt

SEQ ID NO: 29 - DNA, 21 bp, Homo sapiens
cgcctcaatcctccctttatc

SEQ ID NO: 30 - DNA, 22 bp, Homo sapiens
ctacacgttgagtaggcgaatc

(19) United States
(12) Patent Application Publication
(10) Pub. No.: US 2017/0204395 A1
(43) Pub. Date: Jul. 20, 2017

(54) ESTABLISHMENT OF CELL LINES WITH ALTERED MITOCHONDRIAL DNA COPY NUMBER

(71) Applicant: UNIVERSITY OF SOUTH ALABAMA, Mobile, AL (US)

(72) Inventors: Mikhail Alexeyev, Mobile, AL (US); Domenico Spadafora, Mobile, AL (US)

(21) Appl. No.: 15/338,117

(22) Filed: Oct. 28, 2016

Related U.S. Application Data
(60) Provisional application No. 62/285,482, filed on Oct. 30, 2015.

(51) Int. Cl. C12N 9/00; C12N 5/00

(52) U.S. Cl. CPC C12N 9/93; C12Y 605/01001; C12N 5/00; C12N 2510/00

(57) ABSTRACT

Reduced mitochondrial DNA copy number has been reported in numerous adverse clinical conditions, yet suitable tools for modulating mitochondrial DNA copy number have not been reported. The present disclosure generally pertains to methods for modulating mitochondrial DNA copy number in cells, cell clones obtained from such methods, and cell cultures obtained by culturing such cell clones.

### CROSS-REFERENCE TO RELATED APPLICATION

This application claims priority to U.S. Provisional Patent Application No. 62/285,482, entitled “Establishment of Cell Lines with Altered mtDNA Copy Number” and filed on Oct. 30, 2015, which is incorporated herein by reference.

### BACKGROUND OF THE INVENTION

In most mammalian cells, mitochondria generate the bulk of ATP required to sustain a plethora of diverse cellular processes. Besides generating ATP, mitochondria also play important roles in intracellular calcium signaling (1), apoptosis (2), reactive oxygen species (ROS) production (3), biosynthesis of haem and iron-sulfur clusters (4, 5), and other functions. Mitochondria are unique among organelles of mammalian cells in that they house genetic information in the form of mitochondrial DNA (mtDNA).

Most mitochondrial functions depend, directly or indirectly, on mtDNA, which places it at the center of mitochondrial physiology. Mutations in mtDNA have been implicated in neurodegenerative disorders (6), cancer (7), diabetes (8) and aging (9). Importantly, alterations in mtDNA copy number can also result in severe disease, such as mtDNA depletion syndromes (10, 11).

Reduction of mtDNA copy number has been reported in mtDNA depletion syndromes (12), in response to mtDNA damage (13), in response to experimental cerebral ischemia/reperfusion (14) upon intragastric administration of ethanol to experimental animals (15), in cancer (16, 17), and in other conditions.

There is a need to increase the availability of tools to engineer cell lines for stable maintenance of altered mtDNA copy number, thereby facilitating studies on understanding cellular effects of changes in mtDNA content, which occur in various pathophysiological conditions. However, no such tools have been reported so far.

### SUMMARY OF THE INVENTION

In certain embodiments, this disclosure pertains to a method for modulating mitochondrial DNA copy number in a mammalian cell, comprising the step of introducing at least one protein involved in mitochondrial DNA replication to a mammalian cell. In some embodiments, the protein involved in mitochondrial DNA replication is DNA ligase. In some embodiments, the mitochondrial DNA copy number is increased by modulation. In some embodiments, the mitochondrial DNA copy number is greater than the clinically normal average. In certain embodiments, the DNA ligase has at least one mitochondrial targeting sequence. In certain embodiments, the DNA ligase lacks mitochondrial targeting sequence. In some embodiments, the mitochondrial DNA copy number is decreased by modulation. In another embodiment, the mitochondrial DNA copy number is less than the clinically normal average, further comprising the steps of: selecting for the DNA ligase-expressing mammalian cell; and selectively inactivating Ligase 3 in the DNA ligase-expressing mammalian cell. In some embodiments, the introduction of DNA ligase is characterized by expression of the DNA ligase. In some embodiments, the DNA ligase is selected from bacteria or virus. In some embodiments, the DNA ligase is Escherichia coli LigA.

In other embodiments, this disclosure pertains to a mammalian cell clone that expresses a modulated mitochondrial DNA copy number, obtainable by a method comprising the step of introducing at least one protein involved in mitochondrial DNA replication to the mammalian cell clone. In some embodiments, the protein involved in mitochondrial DNA replication is DNA ligase. In some embodiments, the mitochondrial DNA copy number is increased. In some embodiments, the mitochondrial DNA copy number is greater than the clinically normal average. In some embodiments, the DNA ligase has at least one mitochondrial targeting sequence. In some embodiments, the DNA ligase lacks mitochondrial targeting sequence. In some embodiments, the mitochondrial DNA copy number is decreased, further comprising the steps of: selecting for said DNA ligase-expressing cell; and selectively inactivating Ligase 3 in said DNA ligase-expressing cell. In some embodiments, the mitochondrial DNA copy number is less than the clinically normal average. In some embodiments, the introduction of DNA ligase is characterized by expression of the DNA ligase. In some embodiments, the DNA ligase is selected from bacteria or virus. In some embodiments, the DNA ligase is Escherichia coli LigA.

In other embodiments, this disclosure pertains to a cell culture comprising a majority of cell clones with modulated mitochondrial DNA copy number. In some embodiments, the mitochondrial DNA copy number is greater than the clinically normal average. In some embodiments, the mitochondrial DNA copy number is less than the clinically normal average. In some embodiments, the cell culture comprises at least 90% of mtDNA copy number modulated cell clones.

### BRIEF DESCRIPTION OF THE DRAWINGS

FIG. 1A is a histogram showing mtDNA content in multiple subclones. FIG. 1B is a picture of a gel containing DNA from multiple clones. FIG. 1C is a histogram showing mtDNA content in multiple subclones. FIGS. 1A and 1B illustrate the effect of LigA on mtDNA copy number in 4B6 cells. In FIG. 1A, 4B6 cells were transduced with a retrovirus encoding LigA without MTS, and mtDNA copy number was determined in four resulting subclones. In FIG. 1B, subclone #11 was transduced with a retrovirus encoding Cre recombinase, and genomic DNA from ten clones was analyzed by PCR for the presence of unexcised Ligase 3 allele (“Lig3”), excised Ligase 3 allele (“ALig3”), p° phenotype (mtDNA and nuclear DNA (nDNA) primers), and for the presence of Ligase A (“LigA”). FIG. 1C illustrates the mtDNA content of various clones compared to parental cells.

FIG. 2A is a picture of a gel containing DNA from multiple subclones. FIG. 2B is a picture of a gel containing DNA from multiple subclones grown in selective and non-selective media. FIG. 2C is a histogram showing mtDNA content from multiple subclones grown in selective and non-selective media. FIG. 2D is a histogram showing the mean mtDNA content from subclones grown in selective and non-selective media. FIG. 2E is a histogram showing mtDNA content from multiple subclones grown in selective and non-selective media. FIGS. 2A through 2E indicate stability of mtDNA inheritance. Clone #3 was re-cloned in either selective medium devoid of uridine and pyruvate (−UP) or in non-selective medium containing uridine and pyruvate (+UP) after 3 weeks of growth in non-selective medium. In FIG. 2A, subclones were grown in selective medium, and in FIG. 2B, subclones were grown in non-selective medium, each of which were tested for the presence of mtDNA by PCR. In FIG. 2C, mtDNA copy number was determined in five clones grown in either selective (−UP) or non-selective (+UP) conditions. In FIG. 2D, means were compared in a not significant, two tailed Student's t-test assuming unequal variance. In FIG. 2E, mtDNA copy number was determined in six selected clones after a 20-day propagation in either selective (UP) or non-selective (+UP) media.

FIG. 3A is a histogram showing mtDNA copy number in multiple subclones, of which a subset were transduced with wild type or mutant mLig3. FIG. 3B is a histogram showing LigA expression and mtDNA copy number is multiple subclones. FIG. 3C is a histogram showing mtDNA copy number is multiple subclones with an accompying DNA gel showing subclones with excised MTS. FIG. 3D is a histogram showing mtDNA copy number in cells with varying MTS and LigA expression. FIGS. 3A through 3D show the effects of LigA mitochondrial import, through different pathways, on mtDNA copy number. In FIG. 3A, transduction with WT, but not catalytically inactive mLig3 restored mtDNA copy number in clones dependent on LigA for mtDNA replication. In FIG. 3B, intracellular levels of the LigA transcript did not directly correlate with mtDNA copy number. In FIG. 3C, LigA targeting to mitochondria through the canonical presequence-dependent pathway restored mtDNA copy number in cells dependent on the non-canonical pathway for LigA import. MTS removal through FRT/FLPo-mediated recombination lead to a reduction in mtDNA copy number. FIG. 3D shows the effects of LigA mitochondrial import, through different pathways, on mtDNA copy number: in original 4B6 cells; in clone #2, which lacks Lig3 and supports mtDNA replication by LigA import through the non-canonical pathway; in clone #2, which was complemented with another copy of the LigA targeted to mitochondria through the canonical pathway; and in complemented LigA clone after deletion of the MTS, which ablates LigA uptake through the canonical pathway.

FIG. 4A is a graph of OCR over time in parental cells. FIG. 4B is a graph of OCR over time in transduced cells. FIG. 4C is a graph of OCR over time in transduced cells. FIG. 4D is a histogram showing doubling time of multiple subclones. FIGS. 4A through 4D show respiration and growth rates. 4B6 cells and clones with LigA-supported mtDNA replication #1, #2, and #4 were transduced with retroviruses encoding either WT or catalytically inactive K510V mutant mLig3. In FIG. 4A, OCR was determined in the parental cells. In FIG. 4B, OCR was determined in the transduced cells. In FIG. 4C, OCR was determined in the transduced cells. FIG. 4D shows the doubling time of the resulting clones. ***, P<0.001, two-way ANOVA with Dunnett's post-hoc test.

FIG. 5A is a picture of a DNA gel showing Lig3 expression in multiple clones. FIG. 5B is a histogram showing mtDNA copy number is multiple clones. FIG. 5C is a histogram showing mtDNA copy number in multiple clones. FIG. 5D is a histogram showing mtDNA copy in transduced cells. FIGS. 5A through 5D indicate that Chlorella virus ligase can support mtDNA replication at reduced copy number. In FIG. 5A, 4B6 cells were sequentially transduced with retroviruses encoding ChVlig and Cre recombinase, and Lig3 excision was tested in thirteen resulting clones. In FIG. 5B, the resulting clones had variable mtDNA copy number as compared to an arbitrarily chosen clone #1. FIG. 5C shows that clones #3, #6, and #13 maintained >90% reduced mtDNA copy number upon 3-week propagation in the selective medium. FIG. 5D shows mtDNA copy number in the original 4B6 cells transduced with Chlorella virus ligase, two clones (#3 and #14), which were derived from the original by Lig3 excision, and in the clone #14 after transduction with either WT or catalytically inactive mLig3.

FIG. 6A is a histogram showing mtDNA copy number in multiple clones. FIG. 6B is a histogram showing mtDNA copy number in multiple clones. FIG. 6C is a picture of a gel showing Lig3 expression level in multiple clones. FIG. 6D is a picture of a gel showing Lig3 expression level in multiple clones. FIGS. 6A through 6D show a method for establishing mouse cell lines with reduced mtDNA copy number. In FIG. 6A, initial screening for mtDNA copy number in 3T3#53 clones expressing LigA, in which Lig3 exon 1 or was targeted with CRISPR-Cas9. In FIG. 6B, initial screening for mtDNA copy number in 3T3#53 clones expressing LigA, in which Lig3 exon 8 or was targeted with CRISPR-Cas9. In FIG. 6C, the relationship between Lig3 inactivation and mtDNA copy number is shown in clones with targeted exon 1. In FIG. 6D, the relationship between Lig3 inactivation and mtDNA copy number is shown in clones with targeted exon 8.

FIG. 7A is a diagram of a retrovirus construct encoding LigA. FIG. 7B is a diagram of a retrovirus construct encoding Cre recombinase. FIG. 7C is a diagram of a retrovirus construct encoding wild-type mLig 3. FIG. 7D is a diagram of a retrovirus construct encoding K510V mutant mLig 3. FIG. 7E is a diagram of a retrovirus construct encoding LigA fused to FRT-flanked MTS. FIG. 7F is a diagram of a retrovirus construct encoding ChVlig. FIG. 7G is a diagram of a plasmid encoding FLPo and mCherry proteins. FIG. 7H is a diagram of a plasmid encoding expression of sgRNAs. FIGS. 7A through 7H illustrate Vector maps. FIG. 7A shows retrovirus encoding E. coli LigA. FIG. 7B shows retrovirus encoding Cre recombinase. FIG. 7C shows retroviruses encoding WT mouse Lig3. FIG. 7D shows retroviruses encoding K510V mutant mouse Lig3. FIG. 7E shows a retrovirus encoding LigA fused to MTS OTC flanked by FRT sites. FIG. 7F shows a retrovirus encoding the Chlorella virus ligase. FIG. 7G shows a plasmid encoding FLPo and mCherry proteins. FIG. 7H shows a plasmid for expression of sgRNAs to either exon 1 or exon 3 of the mouse Lig3. (Abbreviations: PGK, RSV, SV40 and U6, corresponding promoters).

FIG. 8 is a histogram showing mtDNA copy number in multiple subclones. FIG. 8 portrays variability of mtDNA copy number in cultured cells. In FIG. 8, 4B6 cells were cloned, and mtDNA copy number was determined in six resulting subclones.

FIG. 9A is a chart showing deletions in the Lig3 exon 1 of a clone. FIG. 9B is a chart showing deletion in the Lig3 exon 1 of a clone. FIG. 9C is a chart showing deletion in the Lig3 exon 1 of a clone. FIG. 9D is a histogram showing mtDNA copy number is multiple clones. FIGS. 9A through 9B indicate that deletions in the Lig3 gene are induced by CRISPR/Cas9. In FIG. 9A, deletions in the Lig3 exon 1 are found in a clone with elevated mtDNA copy number. In FIG. 9B deletions in the Lig3 exon 1 are found in clones with reduced mtDNA copy number. In FIG. 9C deletions in the Lig3 exon 8 are found in clones with reduced mtDNA copy number. gRNA targets are underlined. Sequences from an allele containing two in-frame deletions are bold and underlined. Ter, premature translation termination, is bold and italicized. AAG, a codon for active site lysine in exon 8 is in bold. In FIG. 9D, reduced mtDNA copy number phenotype is stable over at least 3 weeks in clones with targeted exon 8. Clones #1 through #4 (FIG. 6B) were grown in media supplemented with uridine and pyruvate, and mtDNA copy number was re-measured.

FIG. 10 is a graph showing mtDNA copy number by clone number at various concentrations of EtBr. FIG. 10 shows that mtDNA depletion is accelerated in clones with reduced mtDNA content. Parental 3T3#52 and its derivatives D4 and E9, in which mtDNA replication is supported by LigA were grown in the presence of indicated EtBr concentrations. A fraction of cells was removed at regular intervals, and mtDNA copy number was determined by qPCR.

### DETAILED DESCRIPTION

Reduced mtDNA copy number has been reported in numerous adverse clinical conditions, yet suitable tools for modulating mtDNA copy number have not been reported. The present disclosure generally pertains to methods for modulating mtDNA copy number in cells, cell clones obtained from such methods, and cell cultures obtained by culturing such cell clones.

As used herein, “clinically normal average” means mtDNA content in the range 40-150% of average.

As used herein: “amp” means bacterial ampicillin resistance gene; “BGH pA” and “SV40 pA” mean the respective viral polyadenylation signals; “ChVlig” means Chlorella virus ligase; “Cre” means bacteriophage P1 Cre recombinase; “F1 ori” means single-stranded origin of replication of the bacteriophage F1; “FRT” means recognition sites for FLP recombinase; “FLPo” means optimized FLP recombinase gene; “GAG” means retroviral GAG protein; “Hph” means hygromycin phosphotransferase, hygromycin resistance gene; “IRES” means internal ribosome entry site; “mLig3 WT” means wild type mouse DNA ligase III; “mLig3K510V” means catalytically inactive mouse DNA ligase III; “LigA” means E. coli DNA ligase A gene; “LTR” means long terminal repeat; “mCherry” means red fluorescent protein mCherry; “MTS” means mitochondrial matrix targeting sequence of human ornithine transcarbamylase; “Neo” means G418 and kanamycin resistance gene; “ori” means bacterial origin of replication; and “WPRE” means woodchuck hepatitis virus posttranscriptional regulatory element.

As used herein, “modulate” means to increase or decrease.

In certain embodiments, the present disclosure pertains to a method for increasing mtDNA copy number in one or more cells, comprising the step of introducing at least one protein involved in mitochondrial DNA replication to such one or more cells. In some embodiments, the protein involved in mitochondrial DNA replication is DNA ligase. The DNA ligase can be characterized by having at least one mitochondrial targeting sequence. Suitable targeting sequences include, but are not limited to, those found in SOD1, cytochrome oxidase subunit VIII, ornithine and transcarbamylase (18). In one embodiment, the DNA ligase lacks mitochondrial targeting sequence. In certain embodiments, the DNA ligase is selected from bacteria or virus. Examples of DNA ligases suitable for use include, but are not limited to, E. coli LigA, and Chlorella virus ligase. Increasing mtDNA copy number is important because it may provide a therapeutic strategy for mitochondrial diseases like LHON (19, 20).

In certain embodiments, the present disclosure pertains to a method for decreasing mtDNA copy number in one or more cells, comprising the steps of: introducing at least one protein involved in mitochondrial DNA replication to a mammalian cell; selecting for the protein-expressing cell (no selection is required in the case of protein transduction); and selectively inactivating the protein in the protein-expressing cell. In some embodiments, the protein involved in mitochondrial DNA replication is DNA ligase. Suitable examples of DNA ligase include, but are not limited to, E. coli LigA and Chlorella virus ligase. Suitable methods of introducing DNA ligase include, but are not limited to, viral transduction, plasmid transfection, electroporation, biolistic delivery, and protein transduction. Introduction of the DNA ligase can be characterized by expression of the DNA ligase. Suitable methods of selection include, but are not limited to applying antibiotics, screening by western blotting, or screening for co-expression of bioluminescent, fluorescent or histochemical marker(s). The DNA ligase can be selected from bacteria or virus.

In certain embodiments, the present disclosure pertains to a mammalian cell clone with increased mtDNA copy number obtainable by a method comprised of introducing at least one protein involved in mitochondrial DNA replication. In some embodiments, the protein involved in mitochondrial DNA replication is DNA ligase. Suitable DNA ligases include, but are not limited to, E. coli LigA and Chlorella virus ligase. The DNA ligase can be selected from bacteria or virus. The DNA ligase can have at least one mitochondrial targeting sequence. In the alternative, the DNA ligase can lack a mitochondrial targeting sequence. The mtDNA copy number can be greater than the clinically normal average.

In the alternative, the present disclosure also pertains to a mammalian cell clone with decreased mtDNA copy number obtainable by a method comprising three steps. The first step is introduction of at least one protein involved in mitochondrial DNA replication to a mammalian cell clone. The protein involved in mitochondrial DNA replication may be DNA ligase. Suitable methods of introduction include, but are not limited to viral transduction, plasmid transfection, electroporation, biolistic delivery, and protein transduction. The introduction of protein involved in mitochondrial DNA replication can be characterized by expression of the protein. The mtDNA copy number can be less than clinically normal average. The protein involved in mitochondrial DNA replication can be selected from bacteria or virus. DNA ligases suitable for use include, but are not limited to, E. coli LigA and Chlorella virus ligase. The second step is selection for a cell expressing the protein involved in mitochondrial DNA replication. Suitable methods of selection include, but are not limited to, applying antibiotics, screening by western blotting, and screening for co-expression of bioluminescent, fluorescent and histochemical marker(s). The third step is selective inactivation of protein involved in mitochondrial DNA replication in the protein-expressing cell. Suitable methods of selective inactivation include, but are not limited to, inducing deletions by introducing site-specific recombinases into cells with conditionally targeted protein-specific alleles, TALEN-, Zinc Finger Nuclease- and CRISPR/Cas9-mediated gene inactivation.

In certain embodiments, the present disclosure pertains to a cell culture obtainable by cultivating at least one cell clone as described hereinabove. The cell culture can contain at least 90% cell clones. The cells contain a mtDNA copy number that is greater than clinically normal average.

### Examples

Herein is evidence for the existence, in mitochondria, of a low-capacity protein import pathway is reported, which facilitates uptake of some proteins lacking conventional matrix targeting sequences, and demonstrates that this pathway can be used to establish cell lines with reduced mtDNA copy number.

### Materials and Methods

### Cells, Viruses and DNA Constructs

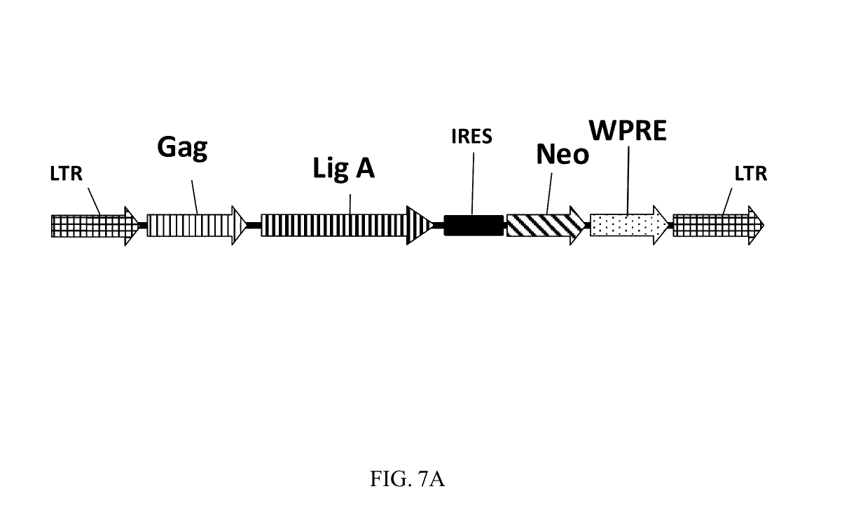

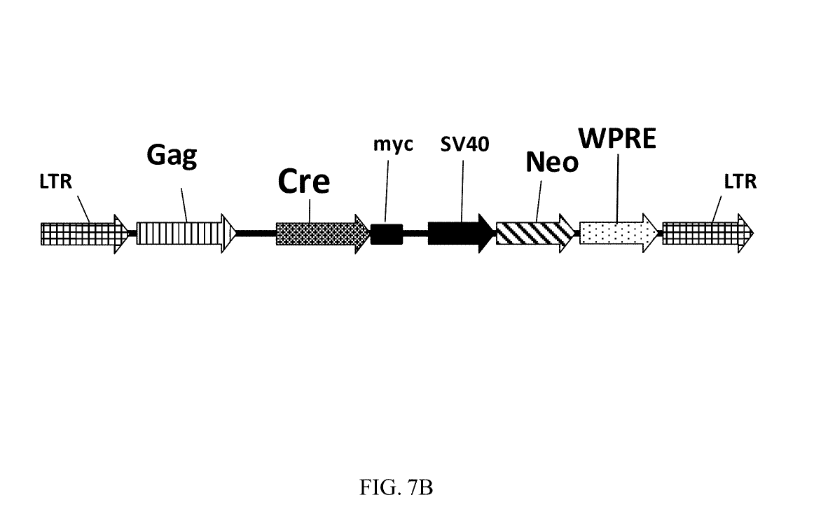

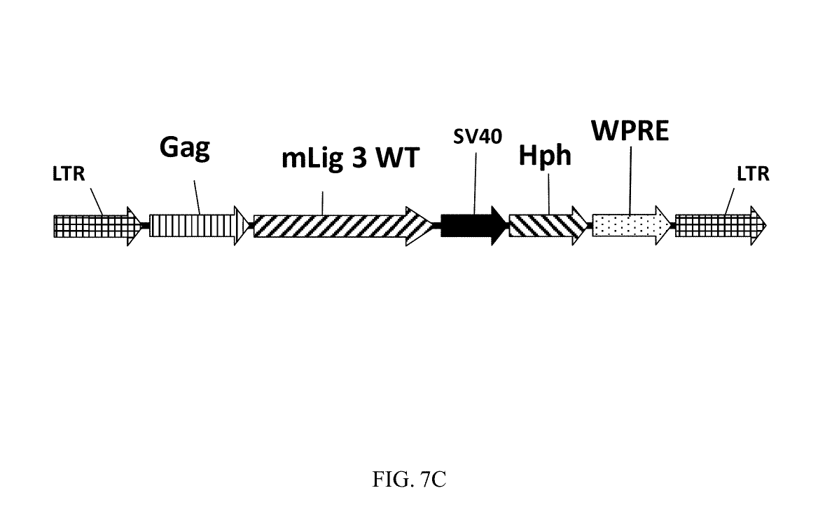

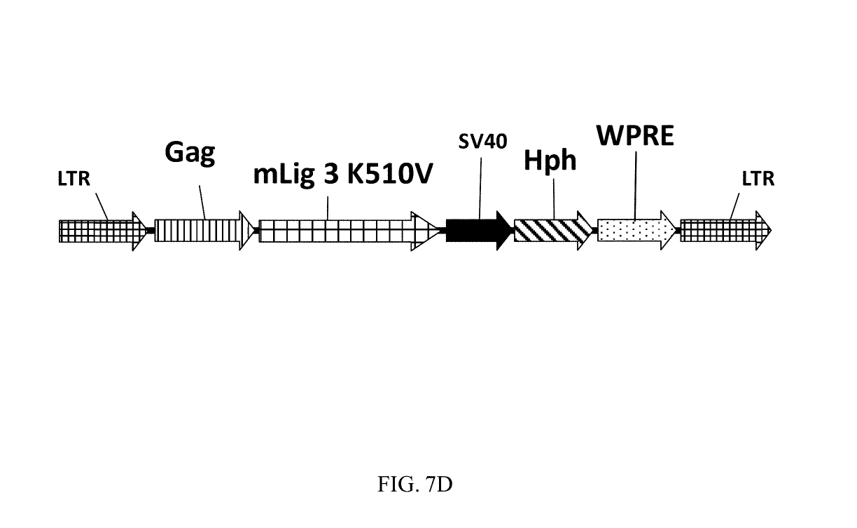

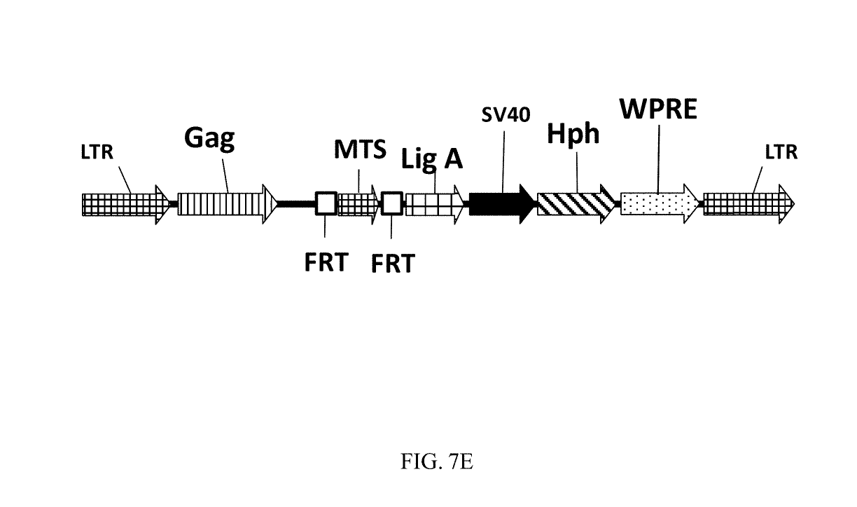

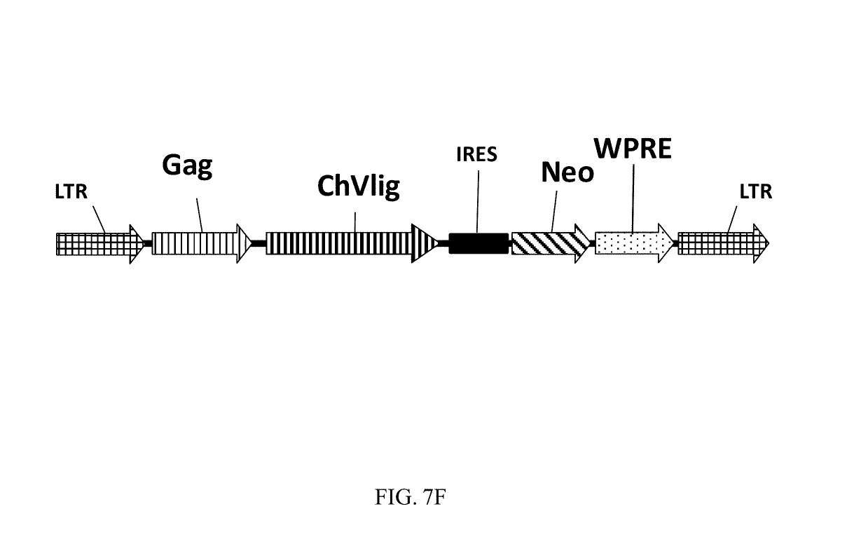

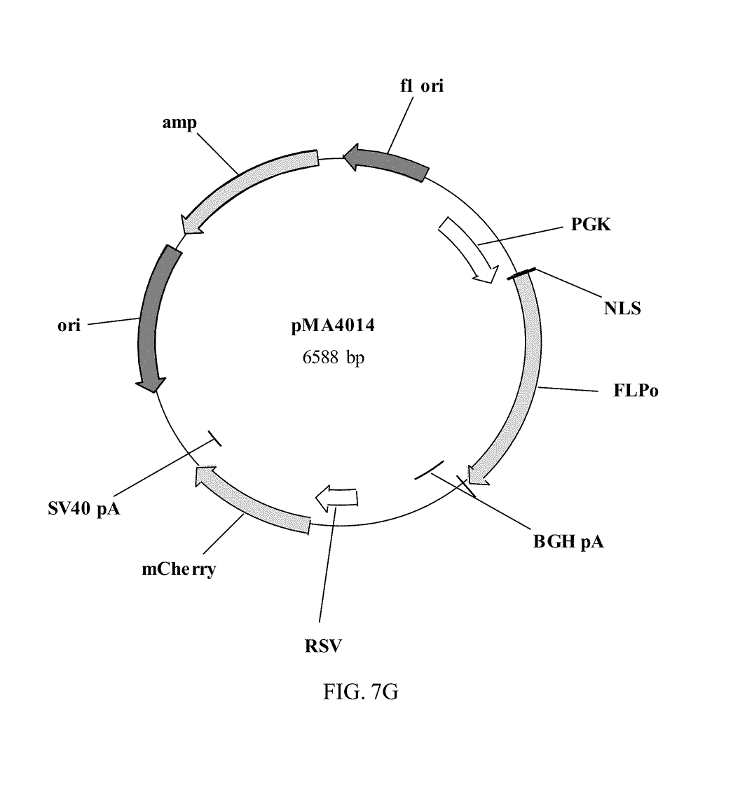

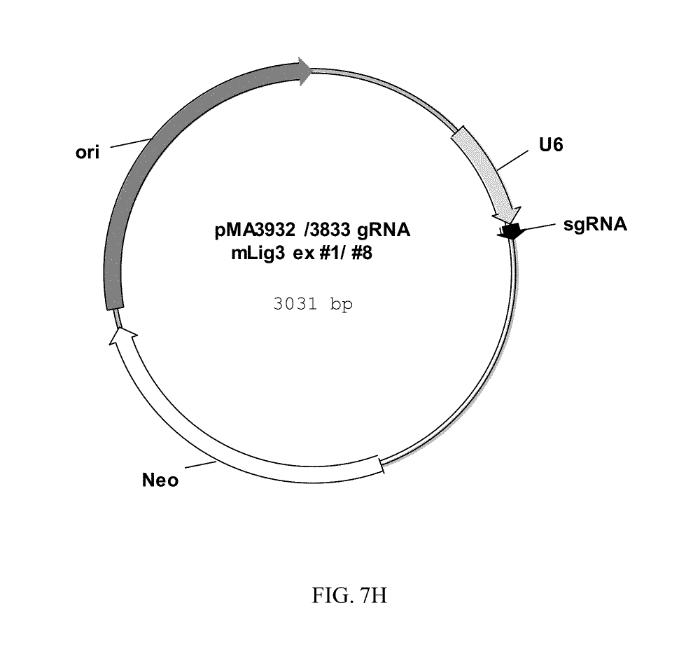

All cells were propagated in Dulbecco's Modified Eagle Medium (DMEM) containing 10% Fetal Bovine Serum, 50 ug/ml gentamycin, 50 ug/ml uridine, and 1 mM sodium pyruvate in a humidified atmosphere containing 5% CO₂ at 37° C., which is permissive for growth of p° cells (+UP medium). When indicated, uridine and pyruvate were omitted from this medium for selection of cells containing mtDNA (−UP medium). 3T3#52 is a Tet-On derivative of the NIH 3T3 cell line (21). 4B6 mouse embryonic fibroblasts were derived from Lig3^{flox/flox} embryos (22). Plasmids and viral constructs were generated by standard techniques (23) and their diagrams are presented in FIGS. 7A through 7G.

### Production of Virus-Containing Supernatants and Infection of Target Cells

Retrovirus-containing supernatants were produced by CaPO₄-mediated transfection of the HEK293FT and Phoenix Ampho cell lines, respectively, using established protocols (24). Target cells were infected with viruses in 24-well plates or in 35-mm dishes at 20% confluence by incubating them overnight with corresponding supernatant in the presence of 10 ug/mL polybrene (Sigma-Aldrich Corp., St. Louis, Mo.). The next day, the supernatant was removed and cells were allowed to recover for 24 h in DMEM, after which cells were trypsinized, transferred into 150-mm dishes, and antibiotic selection (G418, 1,000 ug/mL; puromycin, 3 ug/mL; hygromycin, 400 ug/mL) was applied for 6 days.

### Western Blotting

Protein extracts from treated and control cells were prepared using lysis solution containing 10 mM Tris-HCl, 1% SDS, lx EDTA-free protease inhibitor cocktail (Roche, Indianapolis, Ind.). Protein concentrations were measured using the BCA assay (Pierce, Rockford, Ill., USA). Proteins were separated by PAAG electrophoresis and transferred to PVDF membranes, blocked and incubated with primary and secondary antibodies using standard techniques (23). Blots were developed with SuperSignal West Pico and exposed to CL-Xposure film (both Pierce). Primary antibodies were α-HSP60 (mitochondrial, BD Biosciences), α-cytochrome oxidase subunit 1 (AbCam), α-Lig3 (BD Biosciences).

Determination of mtDNA Copy Number

Determination of mtDNA copy number in mouse cells was performed using duplex TaqMan qPCR essentially as described previously (22) using EcoRl-digested total cellular DNA and primers and probes described in Table 1. To generate standard curves, a separate linearized calibrator plasmid containing cloned nuclear and mitochondrial targets was used. The 20×qPCR primer master mix contained 3.3 uM mitochondrial forward and reverse primers, 1.65 uM mitochondrial probe, 13.2 uM nuclear forward and reverse primers, and 6.6 uM nuclear probe.

### CRISPR-Cas9 Mediated Inactivation of Lig 3

For inactivation of the mouse Lig3 gene by introducing deletions into either exon 1 or exon 8, four plasmids encoding gRNAs were constructed, two for each exon (Table 1 and FIG. 7H). After transducing 3T3#52 cells with a retrovirus encoding LigA without MTS, resultant transductants were transfected with four plasmids encoding Cas9, two gRNA plasmids for either exon 1 or exon 8, and EGFP, respectively. EGFP-positive cells were sorted using FACS, plated, and the resulting colonies were pre-screened by PCR as described previously (25). Clones that appeared positive on PCR screen were tested by western blotted for Lig3, and mtDNA copy number was determined in candidate clones by qPCR.

### Determination of the Borders of CRISPR-Cas9 Induced Deletions

Total DNA was extracted from clones, subjected to PCR with primers Ex1F2 plus Ex1R and Ex8F2 plus Ex8R for exons 1 and 8, respectively (Table 1). PCR products were cloned into pBluescriptII SK+(Stratagene, La Jolla, Calif.), and transformed into E. coli. Inserts were amplified from twelve E. coli colonies for each 3T3 clone with primers dPvu2 and dPvu3 (Table 1), and PCR products were sequenced using primers Ex1F2 or Ex8F2 for exons 1 and 8, respectively. Sequence alignment was performed using CLUSTALW, and edited using MS Word.

### Cellular Respiration

Cellular respiration in whole attached cells was measured with the help of an XF-24 extracellular flux analyzer (Seahorse Biosciences, Billerica, Mass., USA) according to the manufacturer's recommendations and expressed as pMol/min/ug protein. ATP-linked respiration was determined with the help of oligomycin (DUG, 5 uM), maximal respiration was induced with Carbonyl cyanide-4-(trifluoromethoxy)phenylhydrazone (FCCP, 1 uM), and non-mitochondrial respiration was determined after injection of rotenone and antimycin A (R+A, 1 uM each).

### Statistical Analysis

Pairwise comparisons were performed using the two-tailed unpaired student's t-test assuming unequal variances. Multiple comparisons were performed using two-way ANOVA followed by Dunnett's test.

### Results

In Cultured Cells, mtDNA Copy Number Varies Over 2-Fold Range

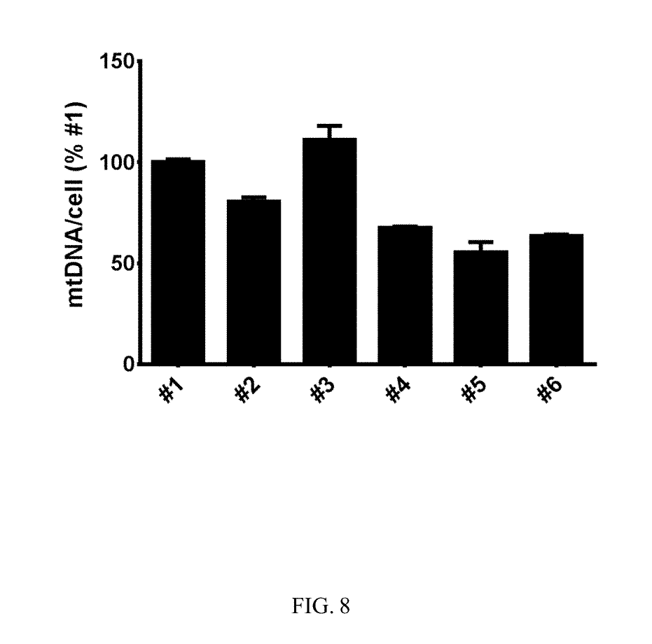

In most population studies, mtDNA copy number in a given tissue in apparently healthy individuals varies over 2-10 fold range (26-29), and mtDNA content in the range 40-150% of the average is considered clinically normal (30). Therefore, Applicants sought to establish mtDNA copy number variability in cultured cells. A population of 4B6 mouse embryonic fibroblasts derived from Lig3^{flox/flox} embryos (22) was cloned, and mtDNA copy number in six resulting clones was determined. It varied over 2-fold range, from 56% to 111% of arbitrary chosen clone #1 (FIG. 8). To confirm this observation, Applicants re-cloned clone #1. Again, mtDNA copy number varied over the similar range.

Switching mtDNA Ligase Alters mtDNA Copy Number

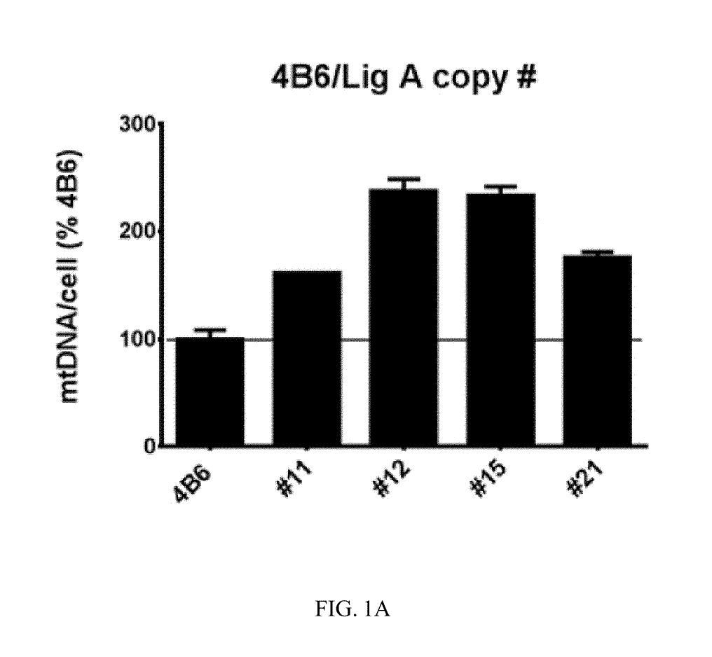

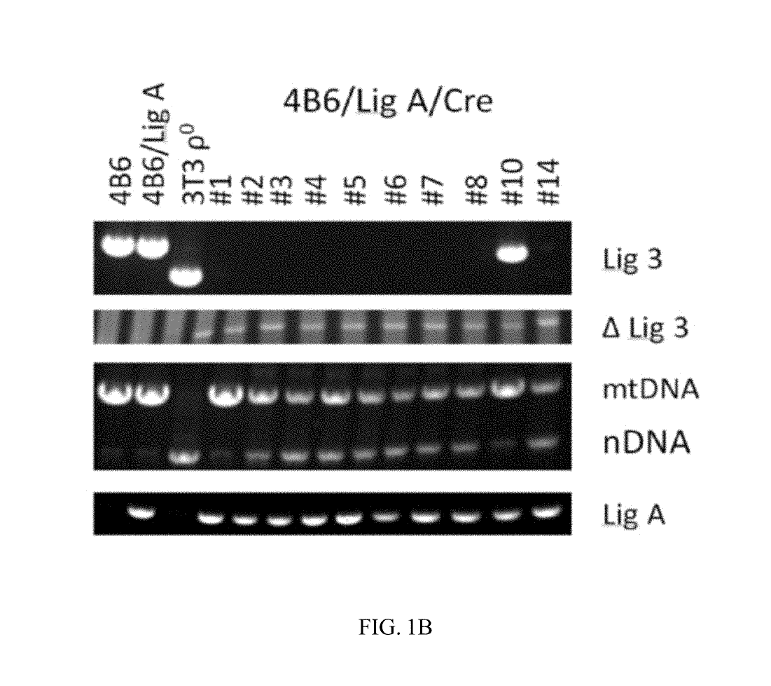

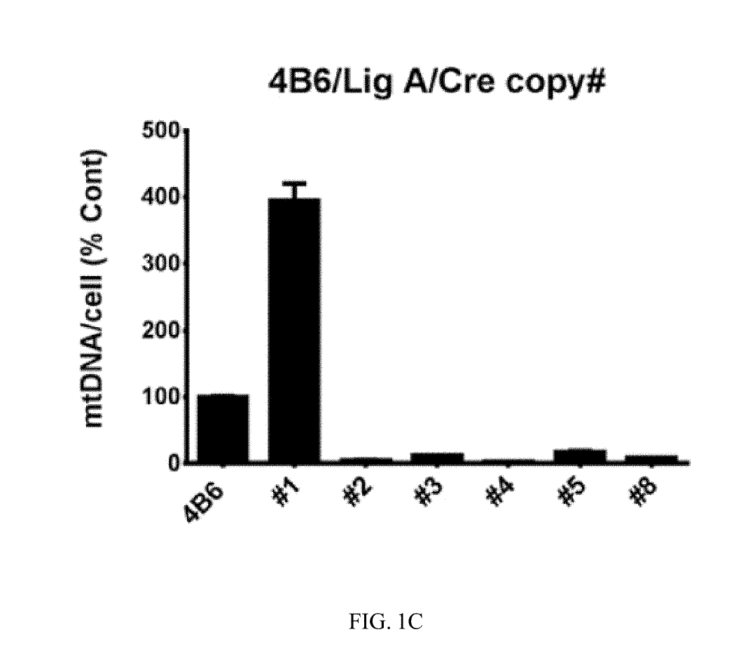

DNA ligase III (Lig3) is the only DNA ligase reported in mitochondria. In cultured cells, the loss of Lig3 is accompanied by the loss of mtDNA without the loss of viability (22). Interestingly, even 100-fold reduced levels of Lig3 in mitochondria are sufficient to maintain mtDNA copy number (22), resulting in the failure of our initial attempts to regulate mtDNA copy number through doxycycline-regulated expression of Lig3 (results not shown). Recently, Simsek et al. reported that both Chlorella virus DNA ligase and E. coli LigA are able to complement Lig3 deficiency even when expressed without mitochondrial matrix targeting sequences (MTS). In silico search for cryptic MTS in these proteins failed to identify potential candidates (results not shown) suggesting that these proteins can be imported through a pathway, which is independent of classical MTS. Because this pathway avoided detection so far, and because most mitochondrial matrix proteins are imported through a canonical MTS-dependent pathway, Applicants hypothesized that this pathway may have low efficiency, and that amount of DNA ligase delivered by this pathway may be insufficient for the maintenance of normal mtDNA copy number. To test this hypothesis, Applicants constructed a retrovirus-encoding LigA gene without an MTS (FIG. 7A) and introduced the construct into 4B6 cells. Unexpectedly, mtDNA copy number in some of the resulting clones was modestly elevated (FIG. 1A). Then, clone #11 (FIG. 1B) was transduced with a retrovirus encoding Cre recombinase (FIG. 7B). PCR analysis of DNA from doubly transduced clones revealed that Cre recombinase efficiently induced deletions in the Lig3 gene so that only one of ten clones tested had incomplete excision (FIG. 1B). Subsequent expansion of the nine clones that underwent a complete deletion in all Lig3 alleles and analysis of mtDNA copy number revealed that two clones lost their mtDNA during expansion and that remaining clones had reduced mtDNA content down to 2% of WT. One clone, clone #1, had over 3-fold elevated mtDNA content as compared to parental cells (FIG. 1C).

FIGS. 1A and 1B show the effect of LigA on mtDNA copy number in 4B6 cells. In FIG. 1A, 4B6 cells were transduced with a retrovirus encoding LigA without MTS, and mtDNA copy number was determined in four resulting subclones. In FIG. 1B, subclone #11 was transduced with a retrovirus encoding Cre recombinase, and genomic DNA from ten clones was analyzed by PCR for the presence of unexcised Lig3 allele (“Lig3”), excised Lig3 allele (“ΔLig3”), p⁰ phenotype (mtDNA and nDNA primers), and for the presence of LigA (“LigA”). Primer sets and primer sequences are listed in Table 1 below.

TABLE 1

| | | | Fragment |
|---|---|---|---|
| Purpose | Name | Sequence | bp |
| Diagnostics of the | Lig3-Del | SEQ ID NO. 1: | 560 |
| mLig3 excision | | CAGTCGACGAGATGGCTCAGTGGT | |
| | | TAAGAGC | |
| | Lig3-5 | SEQ ID NO. 2: | |
| | | GATGCGGCCGCAGCCAAGTGTGAA | |
| | | TATACAGC | |
| Diagnostics of the | Lig3-5 | SEQ ID NO. 3: | 555 |
| mLig3^{flox/flox} | | GATGCGGCCGCAGCCAAGTGTGAA | |
| | | TATACAGC | |
| | Lig3-8 | SEQ ID NO. 4: | |
| | | CAGTCGACAGGGAGCTTGGGACGG | |
| | | ATGC | |
| Lig3 exon 1 sgRNA#3F | Ex1#3F | SEQ ID NO. 5: | N/A |
| | | ACCGTAAAGGGCGTGTGCCGCAT | |
| Lig3 exon 1 sgRNA#3R | Ex1#3R | SEQ ID NO. 6: | |
| | | AAACATGCGGCACACGCCCTTTA | |
| Lig3 exon 1 sgRNA#4F | Ex1#4F | SEQ ID NO. 7: | N/A |
| | | ACCGCATGTTTGAGAAACTGGAA | |
| Lig3 exon 1 sgRNA#4R | Ex1#4R | SEQ ID NO. 8: | |
| | | AAACTTCCAGTTTCTCAAACATG | |
| Lig3 exon 8 sgRNA#3F | Ex1#3F | SEQ ID NO. 9: | N/A |
| | | ACCGTACGATGGTGAGCGAGTCC | |
| Lig3 exon 8 sgRNA#3R | Ex1#3R | SEQ ID NO. 10: | |
| | | AAACGGACTCGCTCACCATCGTA | |
| Multiplex diagnostics | Ex1R | SEQ ID NO. 11: | |
| of CRISPR-Cas9 | | CCAAGAAGGATGCACAGAGAAA | |
| induced deletions in | Ex1F1 | SEQ ID NO. 12: | 311 |
| Lig3, exon 1 | | GTAAAGGGCGTGTGCCG | |
| | Ex1F2 | SEQ ID NO. 13: | 476 |
| | | GCTCTCCAGAGAGGTCATCTAA | |
| | Ex1F3 | SEQ ID NO. 14: | 230 |
| | | ACATTAAGTGCATGTTTGAGAAAC | |
| | | TG | |
| Multiplex diagnostics | Ex8R | SEQ ID NO. 15: | |
| of CRISPR-Cas9 | | TTGTCTCAGGCAGCTCTTTC | |
| induced deletions in | Ex8F1 | SEQ ID NO. 16: | 331 |
| Lig3, exon 8 | | CAAGTACGATGGTGAGCGAG | |
| | Ex8F2 | SEQ ID NO. 17: | 418 |
| | | CATTTGCTTTCTCCATCCCAAG | |
| | Ex8F3 | SEQ ID NO. 18: | 281 |
| | | GCTACTTCAGCCGCAGT | |
| Colony amplification | dPvu2 | SEQ ID NO. 19: | |
| for sequencing | | TGAGCGAGGAAGCGGAAGAG | |
| deletions in Lig3 | dPvu3 | SEQ ID NO. 20: | |
| | | TCAGGCTGCGCAACTGTTGG | |
| LigA qPCR | F | SEQ ID NO. 21: | 95 |
| | | TGCCAGTGAGTTGACCTTAATC | |
| | R | SEQ ID NO. 22: | |
| | | AAGAAGTGCTGGCGTTCTATC | |
| LigA cloning | E.c. LigF | SEQ ID NO. 23: | 2039 |
| | | GCGAATTCGCCACCATGGAATCAA | |
| | | TCGAACAACA | |
| | E.c. LigR | SEQ ID NO. 24: | |
| | | GCTCTAGAGTCAGCTACCCAGCAA | |
| | | ACGCA | |
| Diagnostics of p⁰ | mMitF | SEQ ID NO. 25: | 1041 |
| phenotype in mouse | | AAAGCATCTGGCCTACACCCAGAA | |
| cells | mMitR | SEQ ID NO. 26: | |
| | | ACCCTCGTTTAGCCGTTCATGCTA | |
| | mNucF | SEQ ID NO. 27: | 636 |
| | | CCACGTGCTCTGTATGAGATT | |
| | mNucR | SEQ ID NO. 28: | |
| | | ATGCTGGCTTATCTGTTCCTT | |
| Diagnostics of the | F | SEQ ID NO. 29: | 497/338 |
| FRT-MTS OTC-FRT | | CGCCTCAATCCTCCCTTTATC | |
| excision | R | SEQ ID NO. 30: | |
| | | CTACACGTTGAGTAGGCGAATC | |

Reduced mtDNA Copy Number can be Stably Inherited in the Absence of Selection

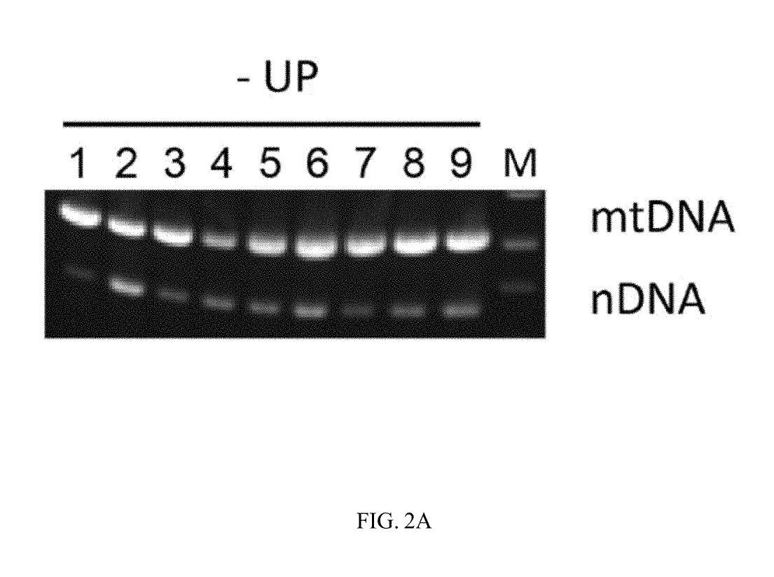

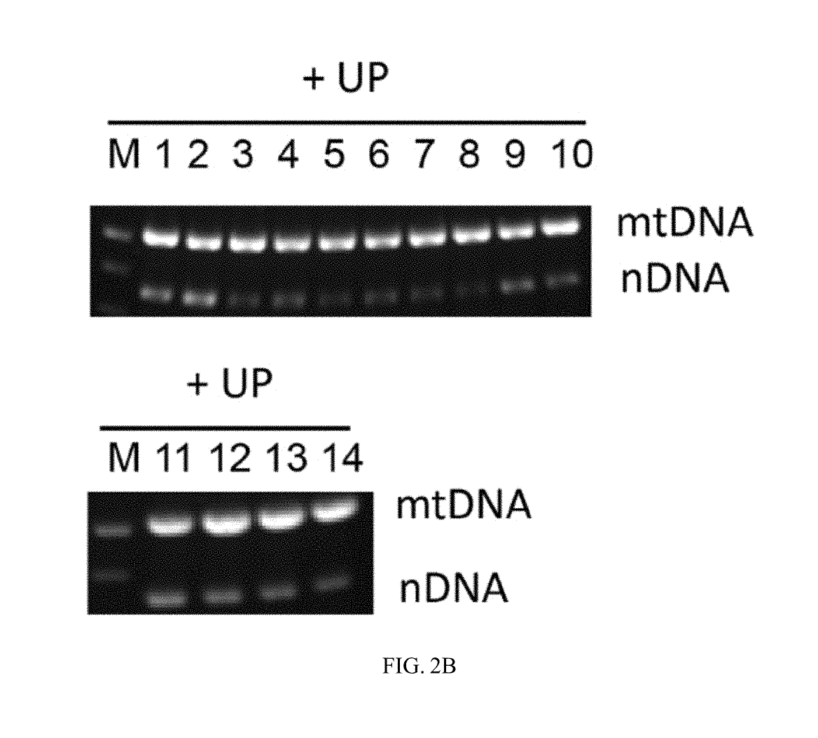

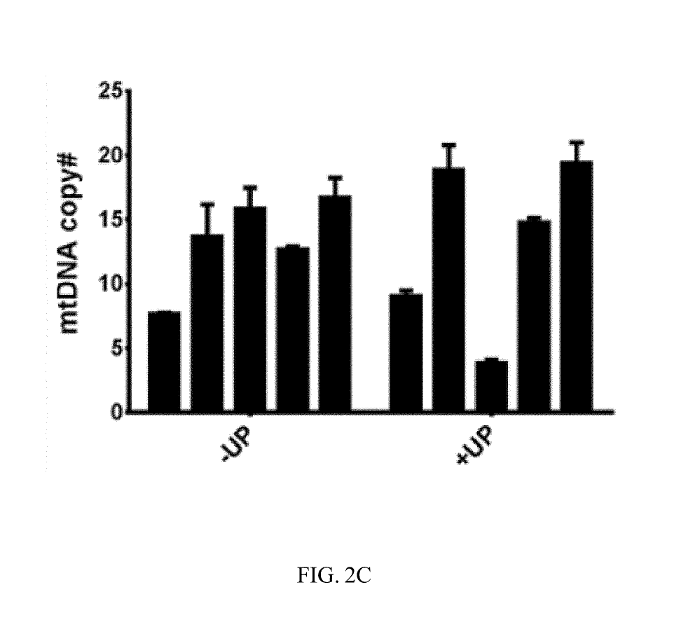

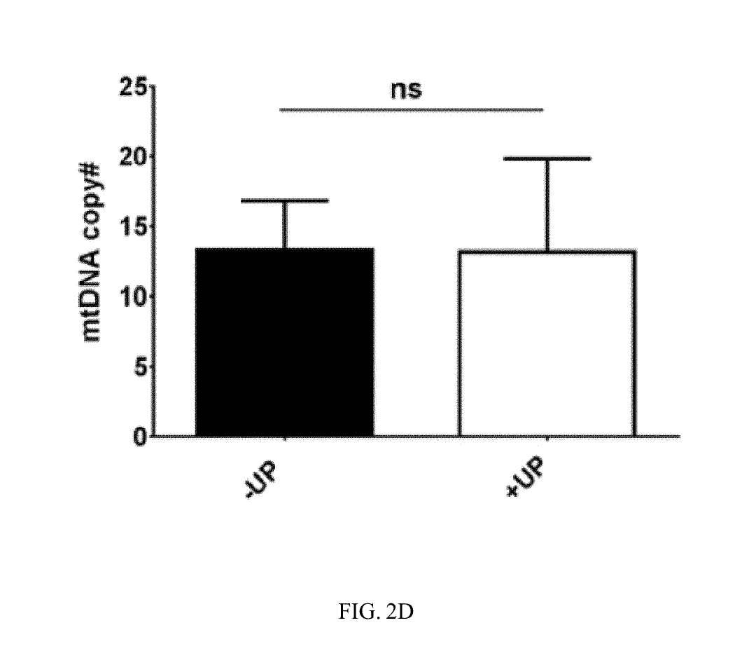

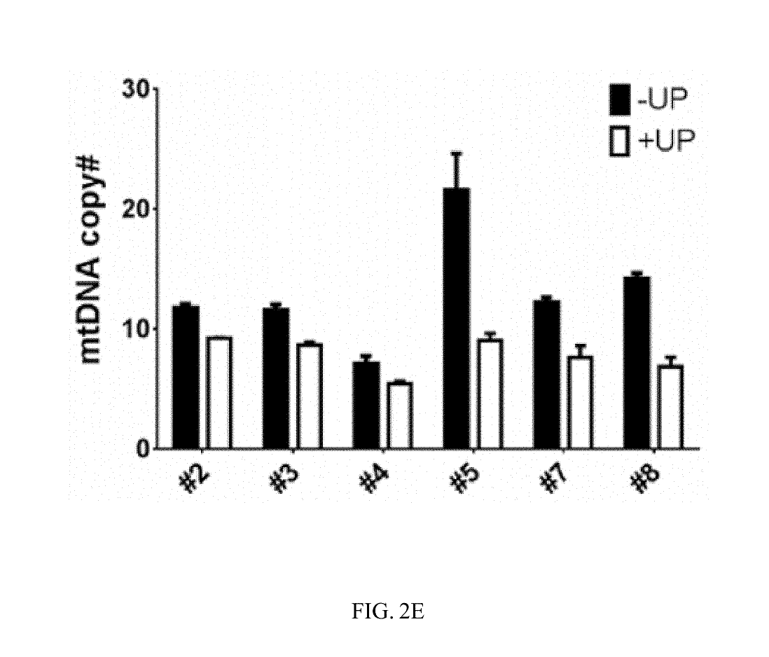

The loss of mtDNA in two clones during expansion in the medium permissive for the growth of the p° cells suggests that in at least some clones with reduced mtDNA copy number LigA-mediated mtDNA maintenance is unstable. This also suggests that low mtDNA copy number observed in most clones may be a consequence of them being mixed populations of cells containing mtDNA and p⁰ cells, with predominance of the latter. To experimentally address this possibility, Applicants re-cloned clone #3, in the complete medium, and in the medium devoid of uridine and pyruvate, which is non-permissive for growth of p⁰ cells. If mtDNA is unstable in clone #3, then during 3 weeks of this clone propagation in non-selective medium prior to cloning, it would be expected to have accumulated a significant fraction of p⁰ cells. In contrast, all clones tested (9 clones grown in selective medium and 14 clones grown in non-selective medium) retained their mtDNA (FIGS. 2A and 2B). Regardless of the medium used for cloning, mtDNA copy number in clones varied (FIG. 2C). However, the average (across all clones tested) mtDNA copy number was not statistically different between clones grown in selective vs. non-selective medium (FIG. 2D).

FIGS. 2A through 2E illustrate the stability of mtDNA inheritance. Clone #3 was re-cloned in either selective medium devoid of uridine and pyruvate (−UP) or in non-selective medium containing uridine and pyruvate (+UP) after 3 weeks of growth in non-selective medium. Subclones grown in either selective (FIG. 2A) or non-selective (FIG. 2B) medium were tested for the presence of mtDNA by PCR. In FIG. 2C, mtDNA copy number was determined in five clones grown in either selective (−UP) or non-selective (+UP) conditions, and means were compared (FIG. 2D). ns, not significant, two-tailed Student's t-test assuming unequal variance. In FIG. 2E, mtDNA copy number was determined in six selected clones after 20-day propagation in either selective (−UP) or non-selective (+UP) media.

A longitudinal study of mtDNA copy number in clones that retained reduced mtDNA copy number during expansion in non-selective conditions was also conducted. Upon propagation in selective vs. non-selective medium for 20 days, mtDNA content was increased in cells grown in selective medium (FIG. 2E). This may indicate preferential growth of cells with increased mtDNA copy number, or low-level instability in the inheritance of mtDNA, which was missed in previous experiments.

mtDNA Copy Number is Dependent on mtDNA Ligase Level

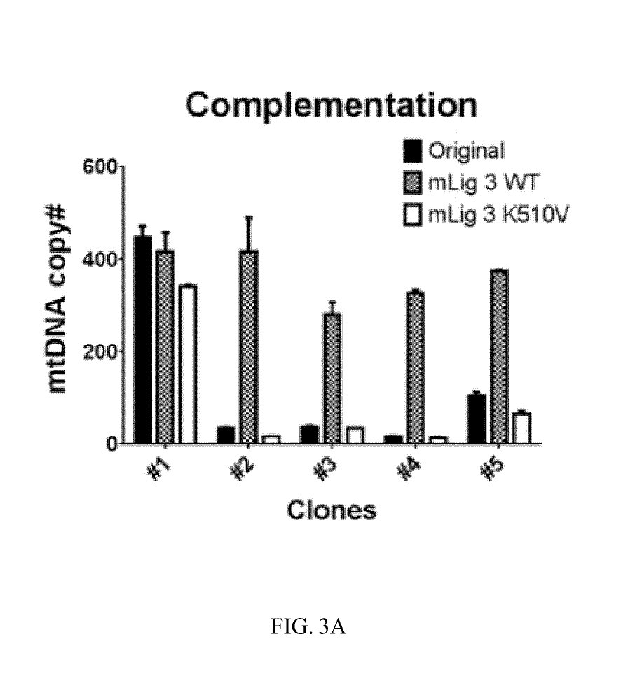

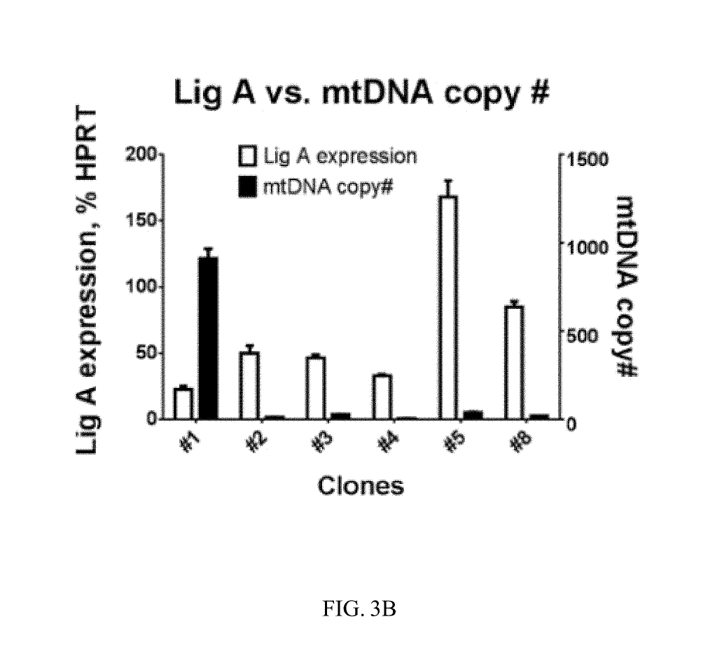

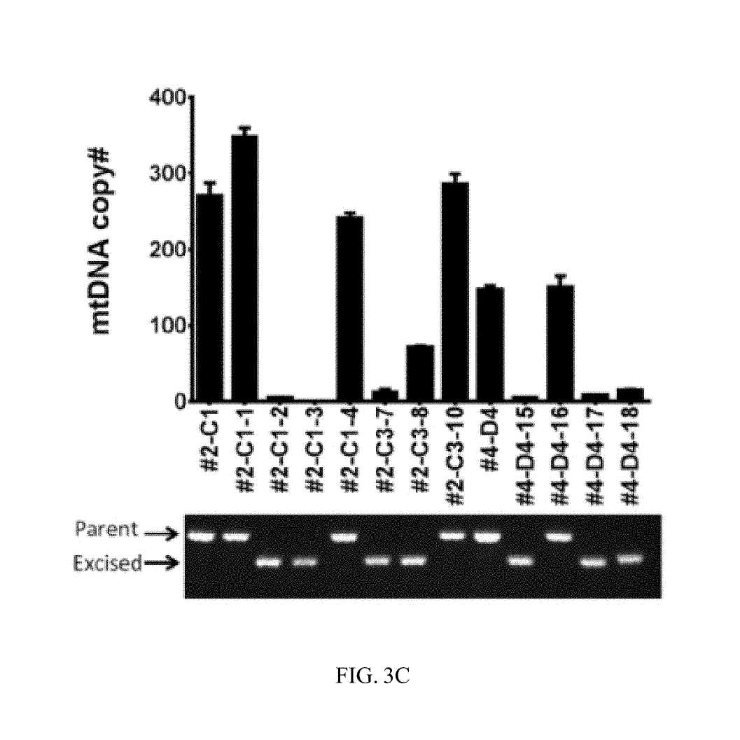

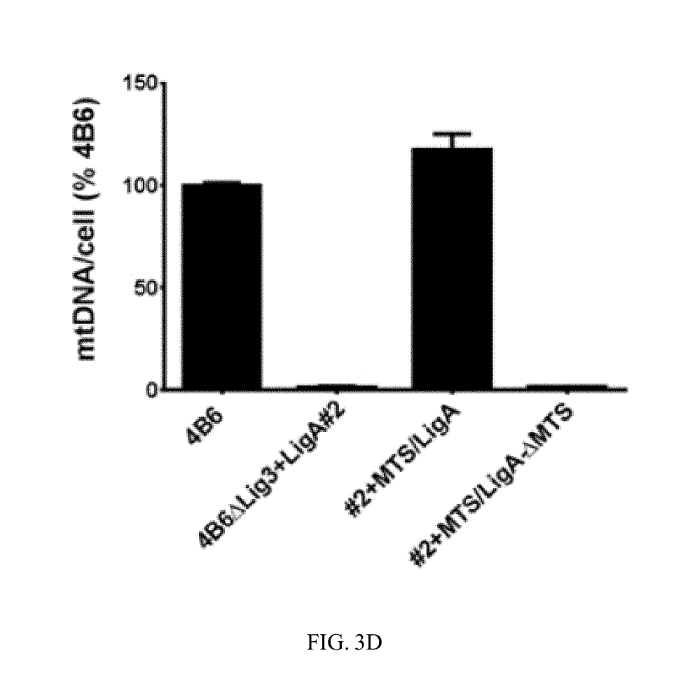

To test whether reduced mtDNA copy number in cells with substitution of the LigA for Lig3 is indeed due to reduced mtDNA ligase availability, Applicants used retroviral transduction to deliver either wild type (WT) or catalytically inactive K510V mutant of the mouse Lig3 (FIGS. 7A-7H) into WT 4B6 cells and into clones with substitution of the LigA for the Lig3. Transduction with WT mLig3 complemented clones with reduced mtDNA copy number, but had little effect on mtDNA content in clone #1, which has high mtDNA content. In contrast, transduction with catalytically inactive mLig3 had little effect on mtDNA content (FIG. 3A). Therefore, reduced mitochondrial content of DNA ligase may be directly responsible for reduced mtDNA copy number.

FIGS. 3A through 3D show the effects of LigA mitochondrial import through different pathways on mtDNA copy number. In FIG. 3A, transduction with WT, but not catalytically inactive mLig3, restores mtDNA copy number in clones dependent on LigA for mtDNA replication. As shown in FIG. 3B, intracellular levels of the LigA transcript did not directly correlate with mtDNA copy number. As shown in FIG. 3C, LigA targeting to mitochondria through the canonical presequence-dependent pathway restored mtDNA copy number in cells dependent on the non-canonical pathway for LigA import. MTS removal through FRT/FLPo-mediated recombination lead to a reduction in mtDNA copy number. FIG. 2D shows effects of LigA mitochondrial import through different pathways on mtDNA copy number: in original 4B6 cells; in clone #2, which lacks Lig3 and supports mtDNA replication by LigA import through the non-canonical pathway; in clone #2, which was complemented with another copy of the LigA targeted to mitochondria through the canonical pathway; and in complemented LigA clone after deletion of the MTS, which ablates LigA uptake through the canonical pathway.

Collectively, observations that (a) LigA is capable of maintaining mtDNA at both high and low copy number and that (b) supplementation with Lig3 restores normal mtDNA levels in clones with low copy number (FIG. 3A) raise the question of whether reduced mtDNA copy number is mediated by low LigA expression or by inefficient mitochondrial import of the LigA, since it lacks a canonical MTS. To distinguish between these two possibilities, qPCR was used to determine both mtDNA copy number and levels of the LigA transcript. Unexpectedly, higher levels of the LigA transcript did not correlate with higher mtDNA copy number (FIG. 3B). This observation is consistent with the hypothesis that mtDNA copy number in these clones is limited by mitochondrial import of the LigA.

To independently confirm that it is inefficient intramitochondrial accumulation of the LigA that is responsible for the reduced mtDNA copy number in clones #2 and #4 (FIGS. 1C, 3A, and 3B), Applicants fused LigA to the MTS from ornithine transcarbamylase (OTC), which was flanked by FRT recombination sites. This fusion can be imported through the efficient canonical, presequence-dependent pathway, and should overcome any limitation that LigA may have in terms of mitochondrial import through a non-canonical pathway. FRT sites facilitate the removal of the MTS OTC through FLP-mediated recombination and thus enable control for positional effects of retrovirus integration when comparing efficiency of mtDNA maintenance by MTS-containing and MTS-devoid versions of the LigA. The resulting construct containing LigA fused to FRT-flanked MTS OTC was inserted into a retroviral vector (FIG. 7E), clones #2 and #4 were transduced with resulting retrovirus, and one subclone from each transduction (2C1 and 4D4, respectively) was selected for transient transfection with a plasmid encoding both optimized FLPo recombinase (31) and red fluorescent protein mCherry (32) (FIG. 7G). Cells expressing mCherry were sorted using FACS and plated to form colonies. In colonies formed by cells in which recombination removed MTS OTC (as judged by PCR with primers flanking this structure), mtDNA copy number dropped as compared to parental cells (FIG. 3C). In contrast, when recombination failed to remove MTS from LigA, the resulting colonies maintained high mtDNA copy number (FIG. 3C). In summary, when in 4B6 cells Lig3 was replaced with LigA lacking MTS, mtDNA copy number dropped (clone #2). When this clone was transduced with LigA fused to MTS, mtDNA copy number was restored. Finally, removal of the MTS in a clone with restored mtDNA copy number due to transduction with LigA fused to MTS resulted in a drop in mtDNA copy number (FIG. 3D). These observations provide a strong support to the notion that reduced LigA import through a non-canonical pathway is directly responsible for reduced mtDNA copy number.

Physiological Effects of Reduced mtDNA Copy Number

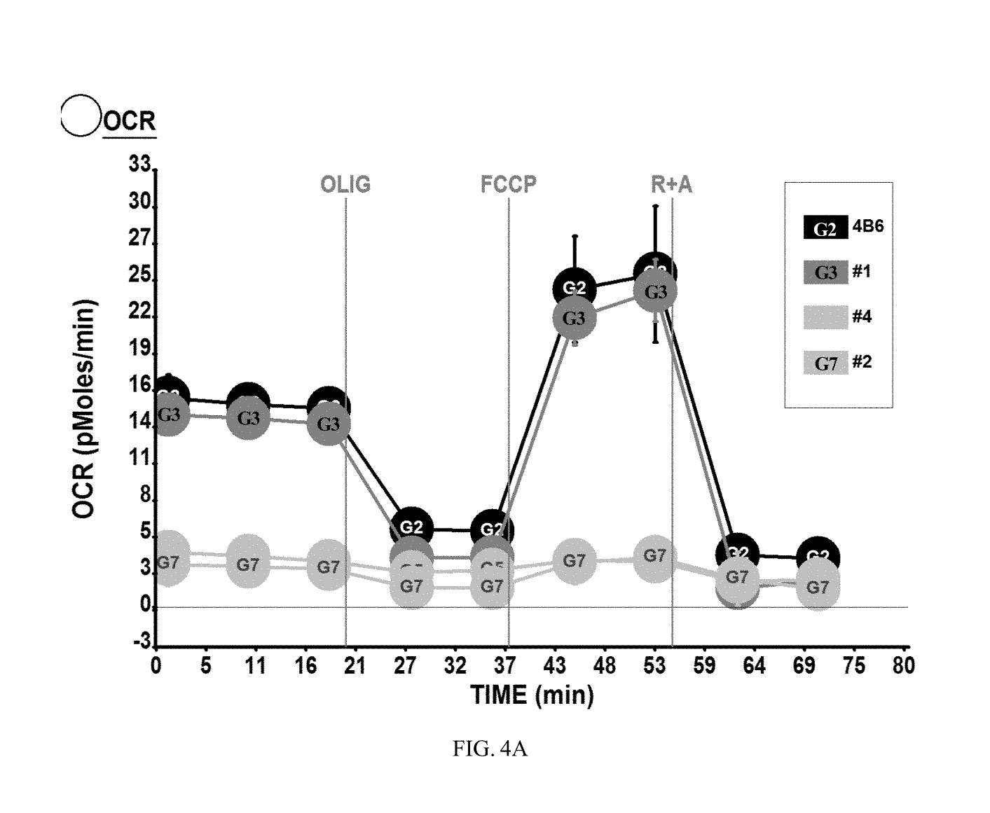

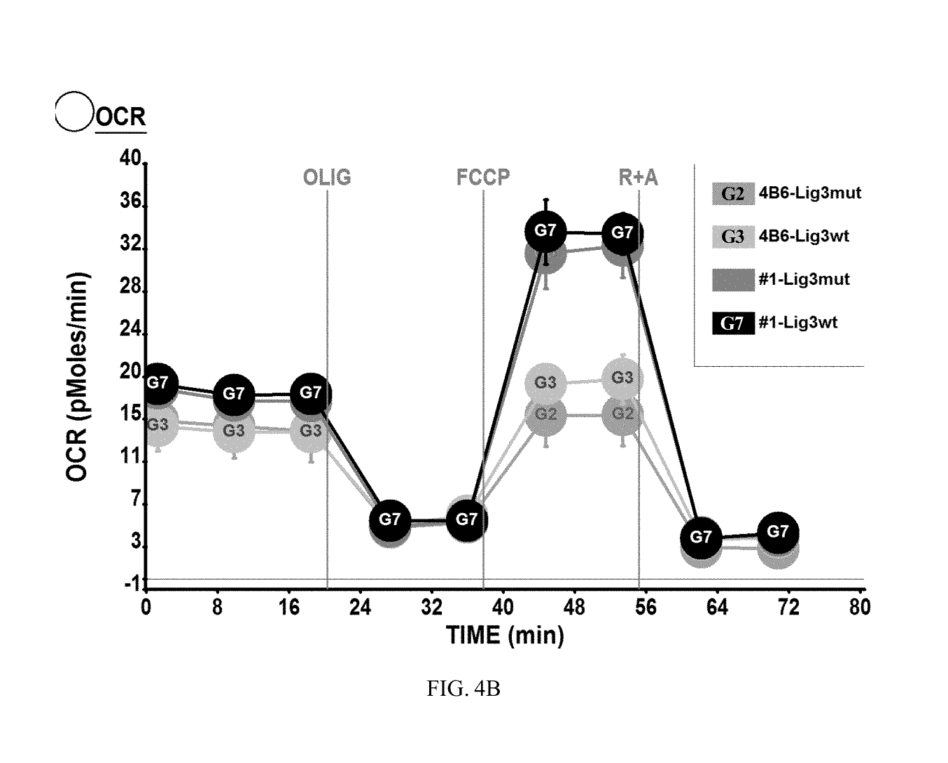

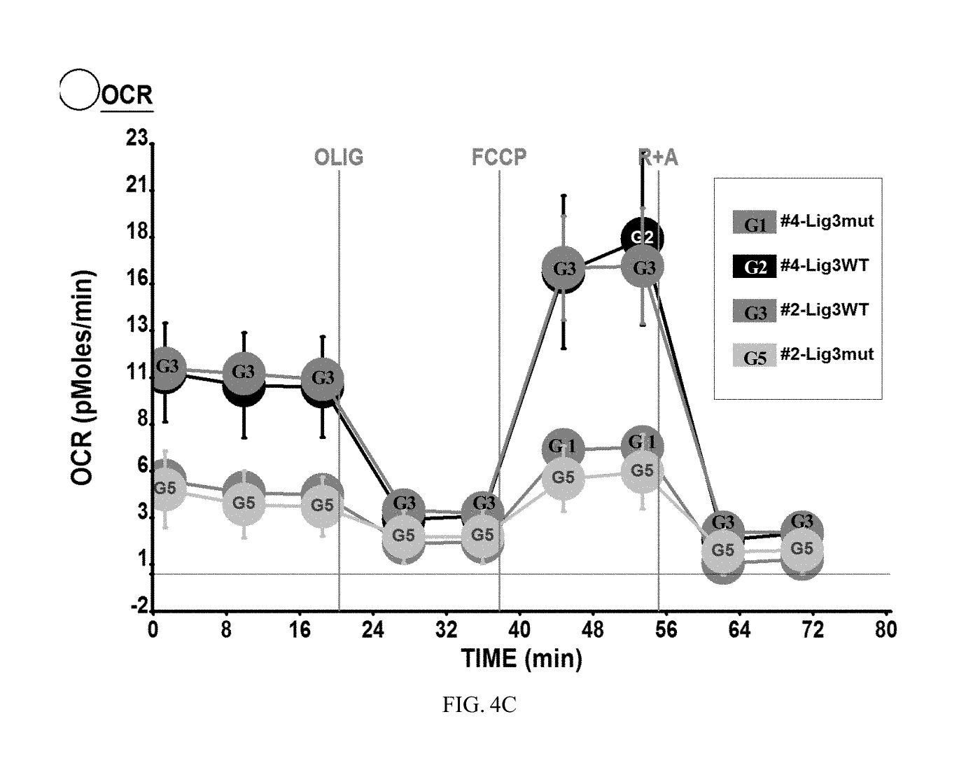

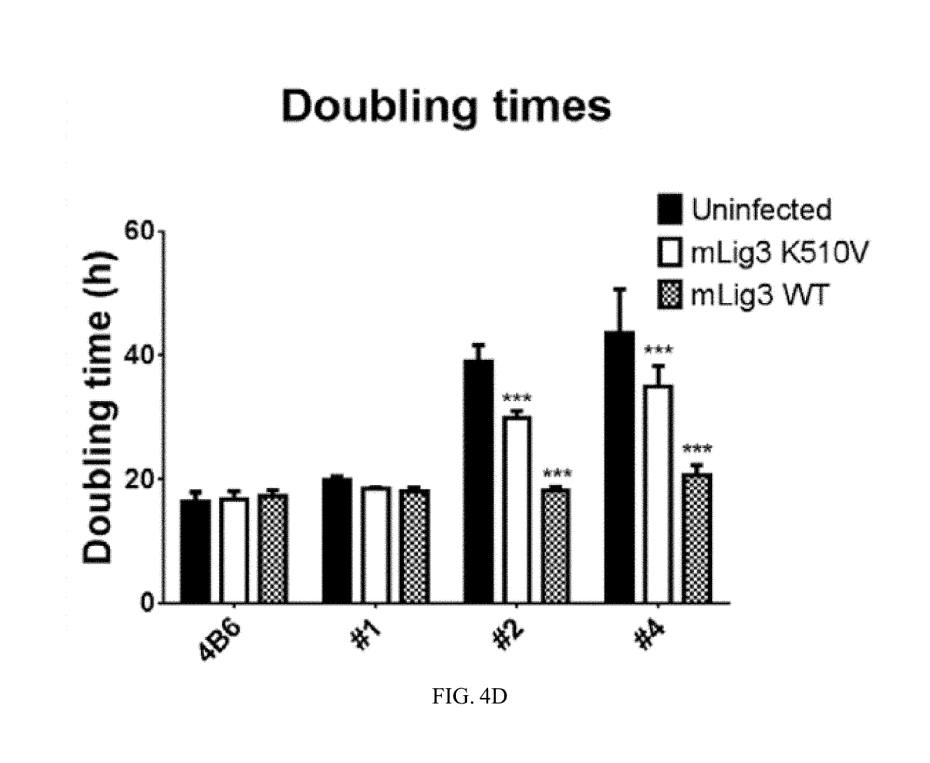

In patients with mtDNA depletion syndromes, mtDNA copy number is severely reduced, and mitochondrial respiratory function is compromised (10). Yet population studies suggest that mtDNA content in tissues of healthy (and therefore, having normal respiratory function) individuals may vary by as much as 10-fold (26-29). Moreover, it has been observed that reduced mtDNA copy number has no major effect on mitochondrial transcript levels or enzyme activities in various tissues (33, 34). Overall, these observations suggest that while, within limits, alterations in mtDNA copy number may have little physiological effect, severe mtDNA depletion is detrimental. Therefore, Applicants set out to investigate physiological consequences of mtDNA depletion in cell lines, in which mtDNA replication is supported by LigA. First, Applicants measured the baseline mitochondrial respiration in WT 4B6 cells and in clones #1, #2 and #4 (FIG. 1C). Despite elevated mtDNA copy number, the oxygen consumption rate (OCR) in clone #1 was the same as in WT 4B6 cells. However, in clones #2 and #4, which have reduced mtDNA copy number, baseline respiration was suppressed as compared to WT 4B6 cells (FIG. 4A). Introducing either WT or K510V mutant mLig3 into either 4B6 cells, or into clone #1 had little effect on the baseline OCR (FIG. 4B). In contrast, transduction of clones #3 and #4 with WT, but not K510V mutant mLig3 led to a significant increase in baseline OCR (FIG. 4C), suggesting that reduced mtDNA copy number is responsible for the reduced OCR. Also, empirical observations of variability in growth rates between parental and virus-transduced cell lines suggested precise measurements of doubling. Doubling times in cell lines were inversely proportional to respiration. Transduction of the WT 4B6 cells and a high mtDNA copy number clone #1 with either WT or mutant Lig3 had no effect on growth rates, whereas transduction of low mtDNA copy number clones #2 and #4 with either WT or, surprisingly, with mutant mLig3 resulted in a significant acceleration of growth (FIG. 4D).

FIGS. 4A through 4D show respiration and growth rates. 4B6 cells and cloned with LigA-supported mtDNA replication #1, #2, and #4 were transduced with retroviruses encoding either WT or catalytically inactive K510V mutant mLig3. In FIGS. 4A through 4C, OCR was determined in the parental (FIG. 4A) and transduced (FIGS. 4B and 4C) cells. FIG. 4D shows doubling time of the resulting clones. ***, P<0.001, two-way ANOVA with Dunnett's post-hoc test.

Chiorella Virus Ligase can Support mtDNA Replication at Reduced Copy Number

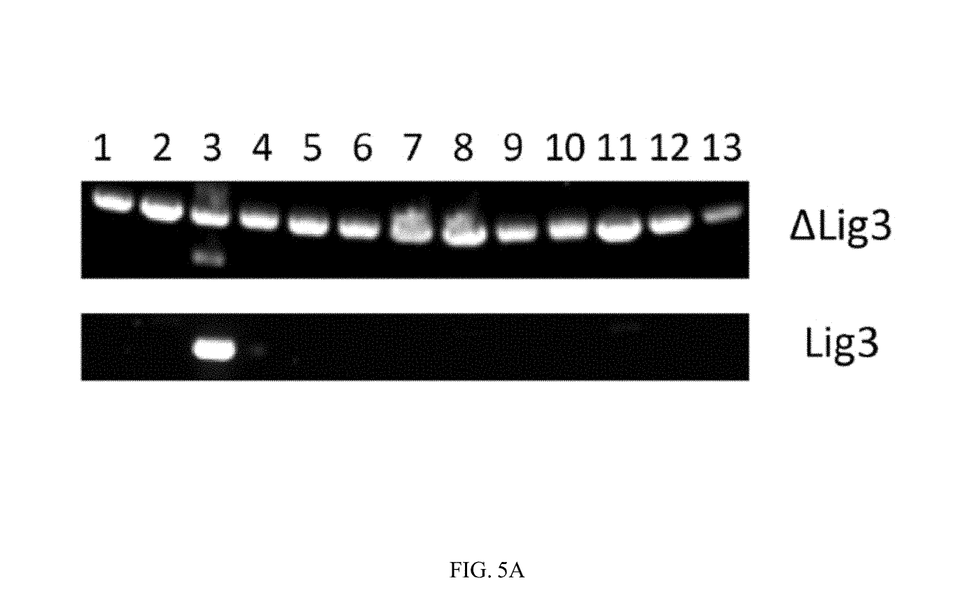

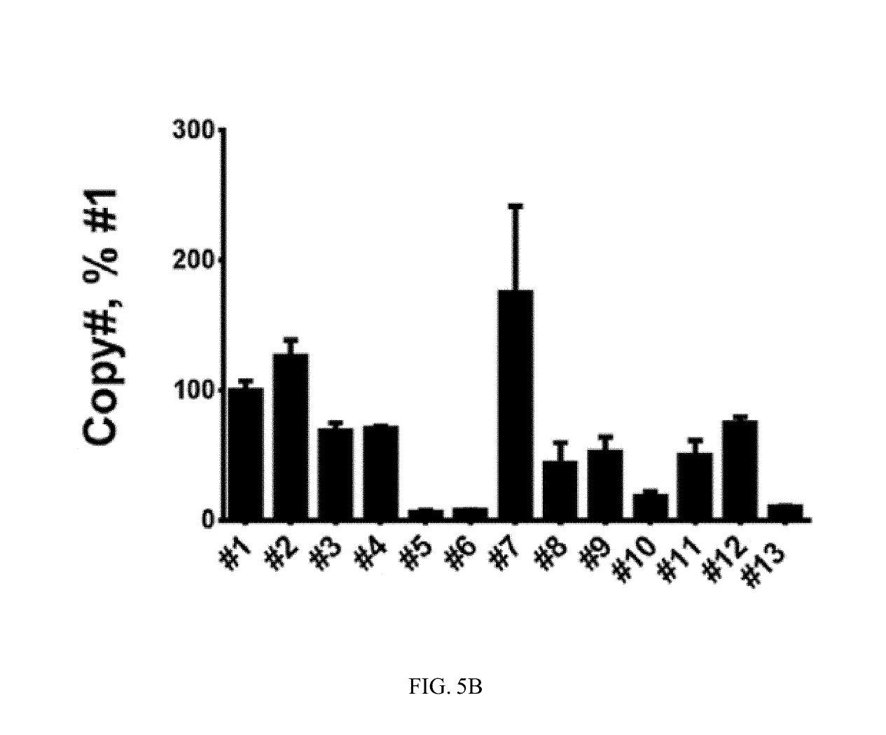

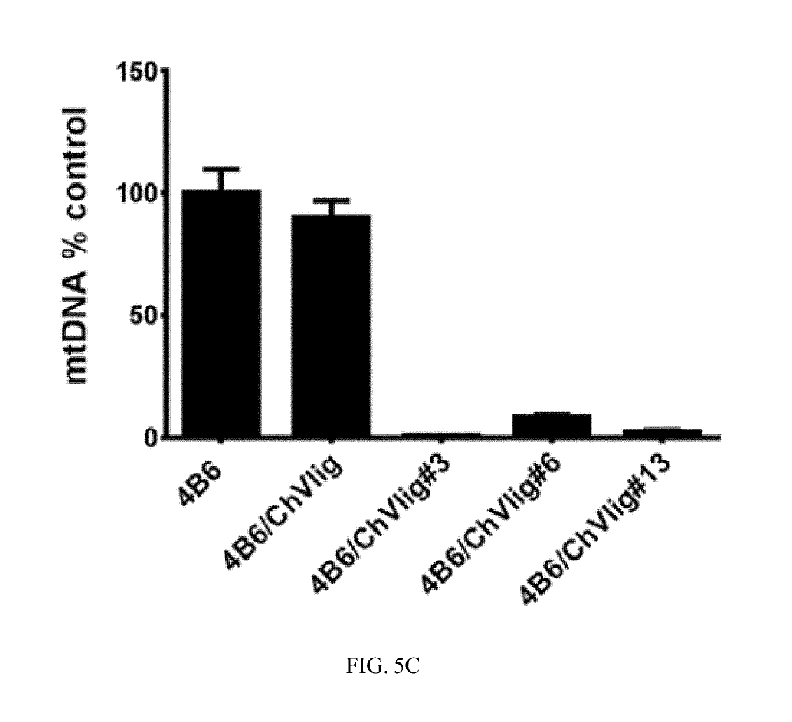

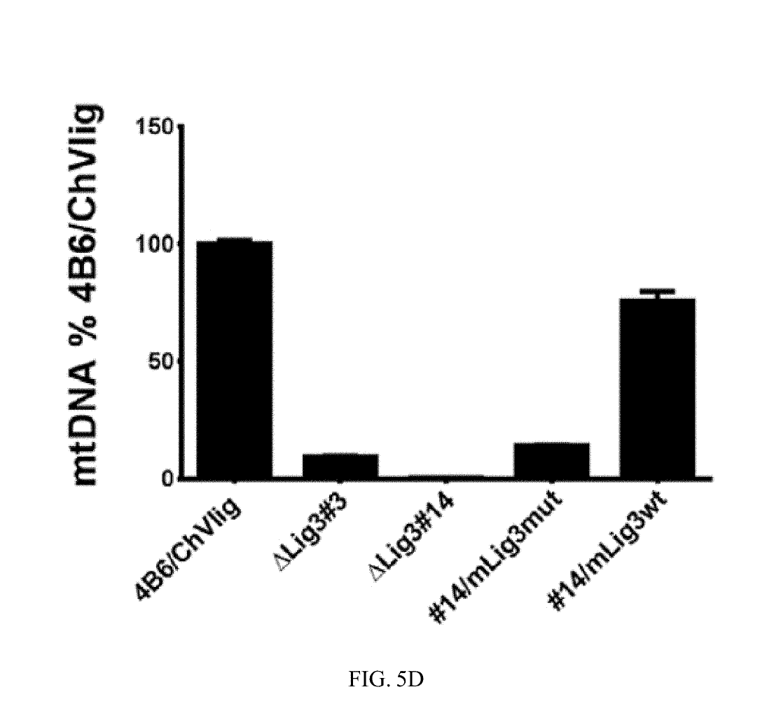

Applicants were further interested in determining whether mitochondrial import of the Chiorella virus ligase (ChVlig), the smallest known eukaryotic DNA ligase, through an MTS-independent pathway supports mtDNA replication at reduced copy number, as does that of LigA. Unlike the LigA, which uses NAD+ as cofactor (35), ChVlig is similar to the Lig3 in that it uses ATP in DNA end-joining reaction (36). 4B6 cells were transduced with a retrovirus encoding Chlorella virus ligase, and one of the resulting clones was re-transduced with a retrovirus encoding Cre recombinase. As in the previous experiment, Lig3 excision was very efficient (12 out of 13 clones tested underwent a complete excision, whereas excision in one clone was incomplete (FIG. 5A). The mtDNA copy number in the resulting clones was variable, and reduced by more than 90% in three clones (FIG. 5B). These clones retained reduced mtDNA copy number upon 3-week propagation in the selective medium (FIG. 5C). Importantly, WT but not catalytically inactive mLig3 was able restore mtDNA copy number in a clone dependent on the ChVlig for mtDNA replication (FIG. 5D). This observation suggests that, similar to LigA, the reduction of mtDNA content may be mediated by inefficient mitochondrial import of ChVlig.

FIG. 5A through 5D show Chlorella virus ligase can support mtDNA replication at reduced copy number. In FIG. 5A, 4B6 cells were sequentially transduced with retroviruses encoding ChVlig and Cre recombinase, and Lig3 excision was tested in thirteen resulting clones. In FIG. 5B, the resulting clones have variable mtDNA copy number as compared to an arbitrarily chosen clone #1. In FIG. 5C, clones #5, #6, and #13 maintained >90% reduced mtDNA copy number upon 3-week propagation in the selective medium. In FIG. 5D, mtDNA copy number in the original 4B6 cells transduced with Chlorella virus ligase, two clones (#3 and #14), which were derived from the original by Lig3 excision, and in the clone #14 after transduction with either WT or catalytically inactive mLig3.

A Method for Establishing Mouse Cell Lines with Reduced mtDNA Copy Number

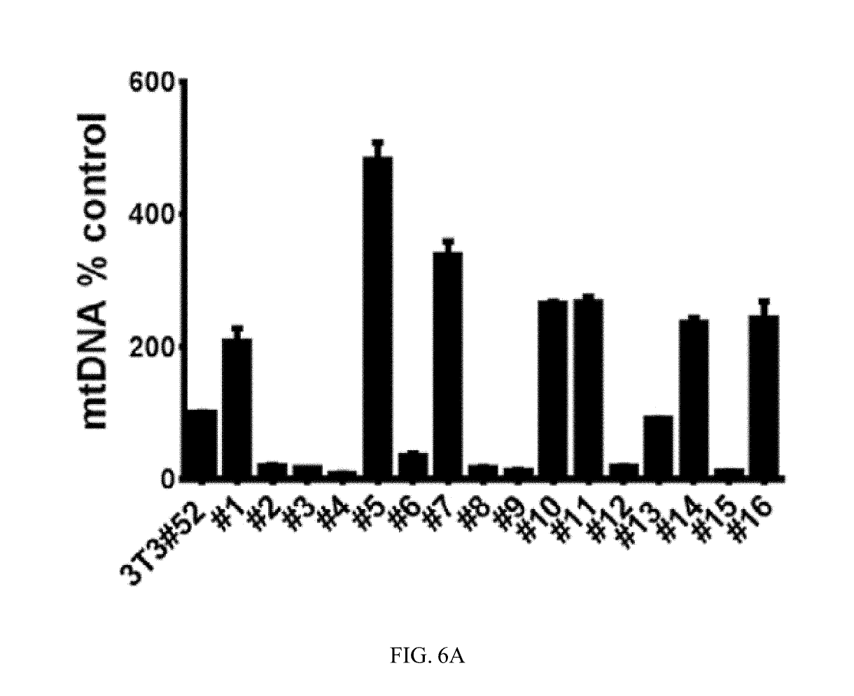

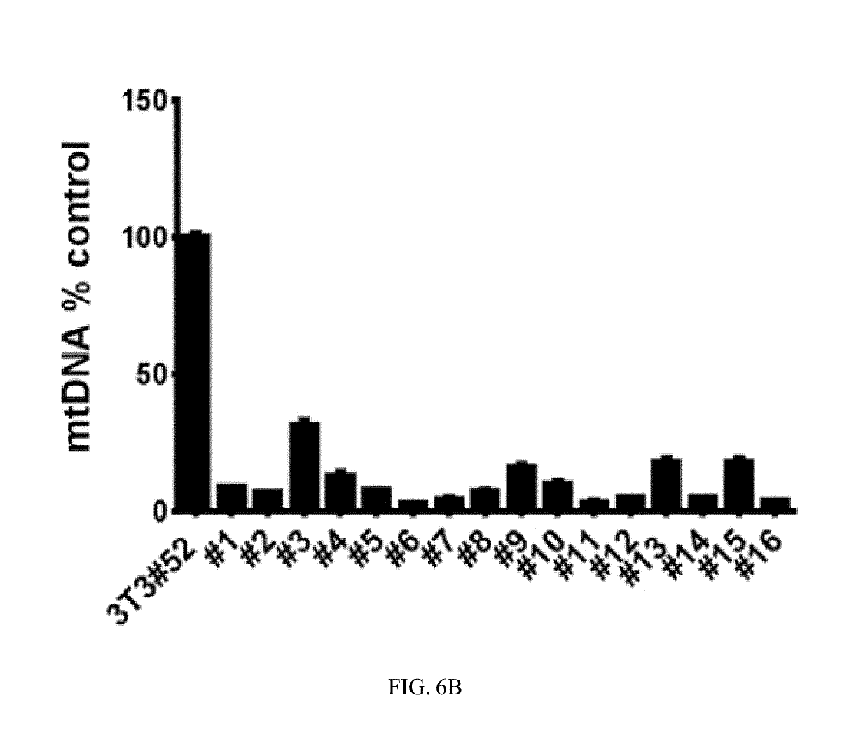

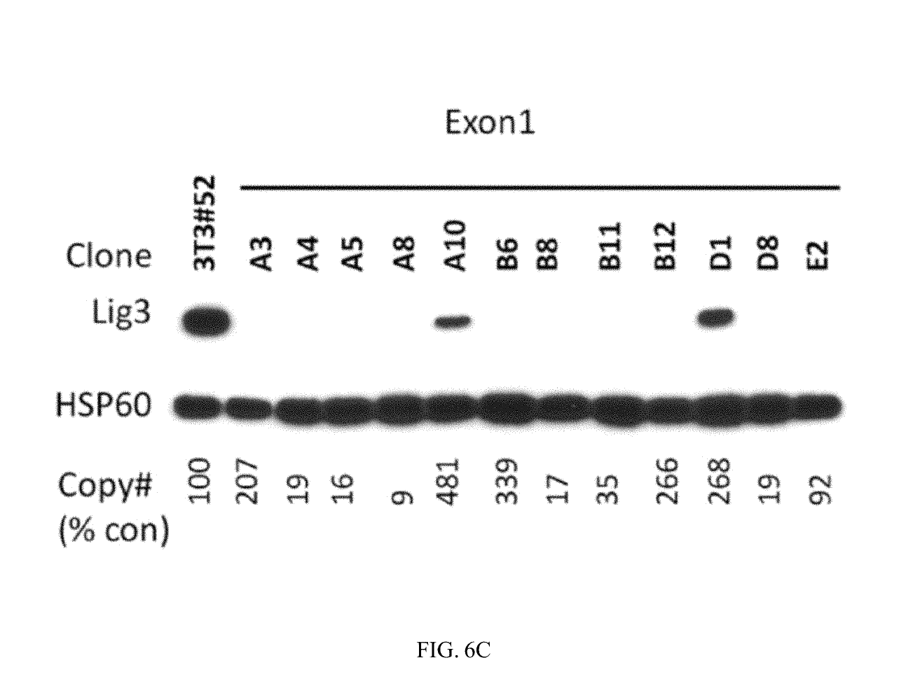

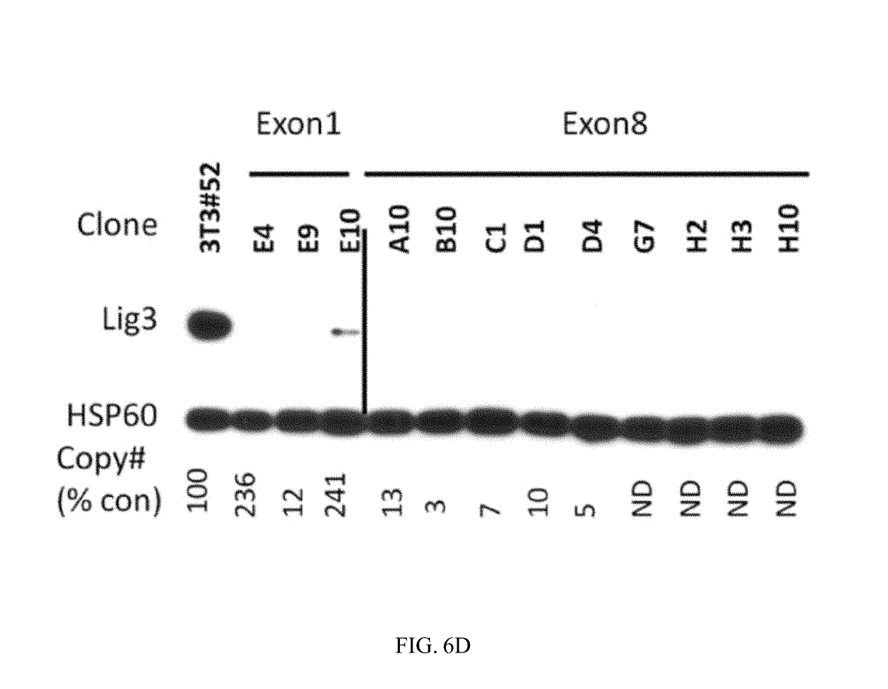

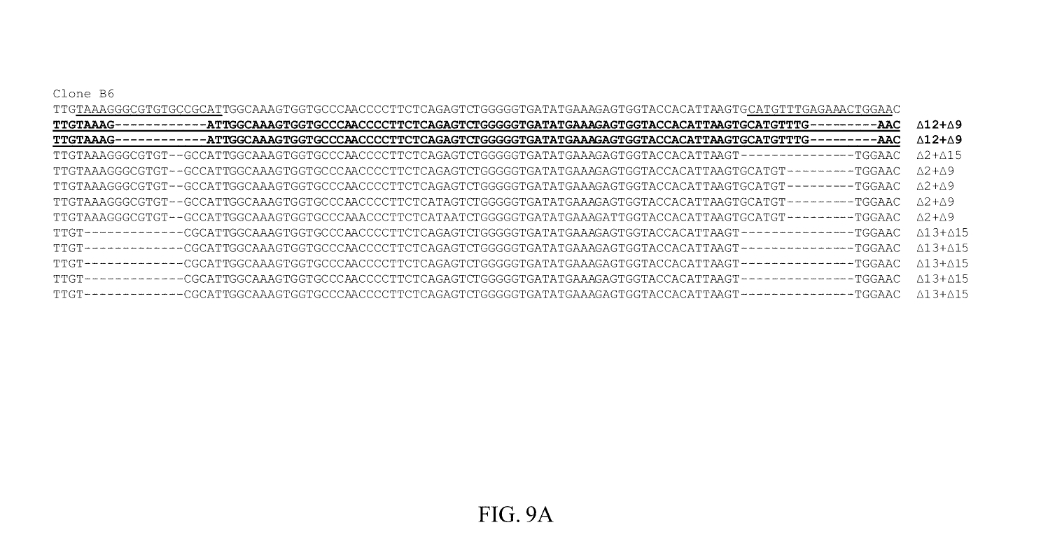

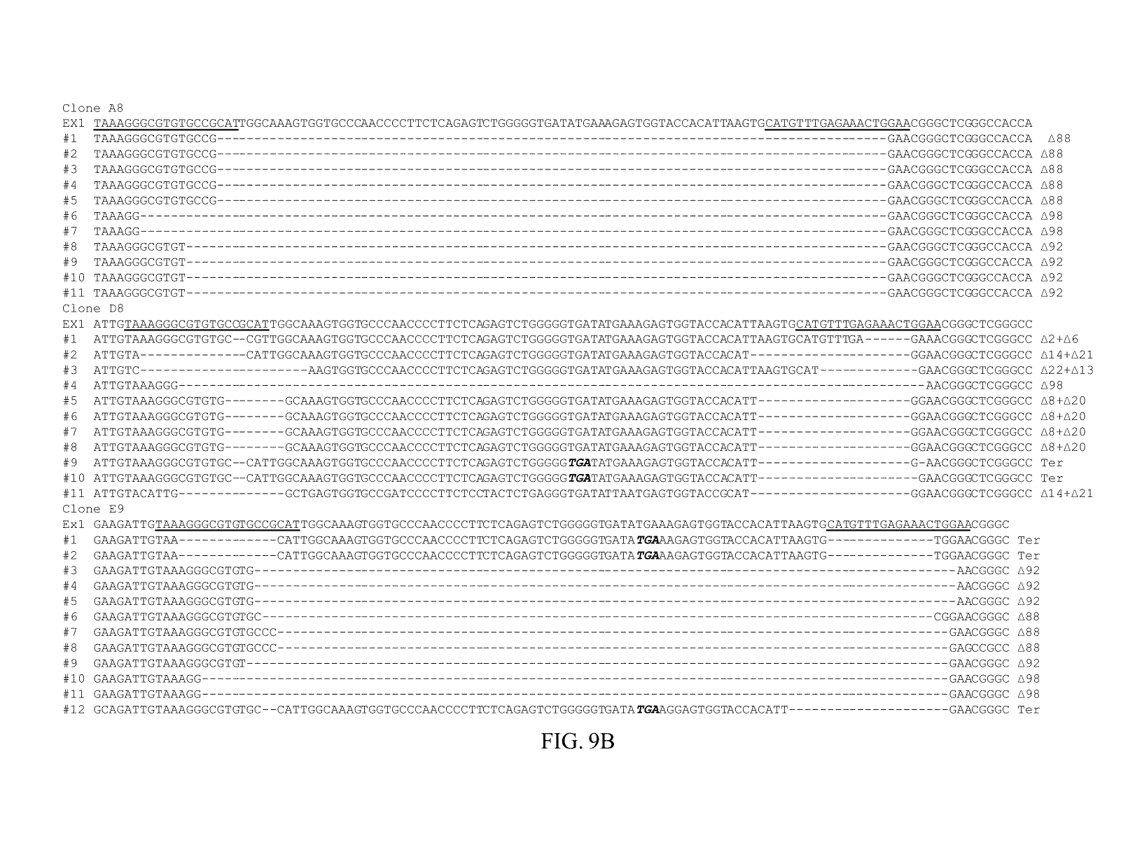

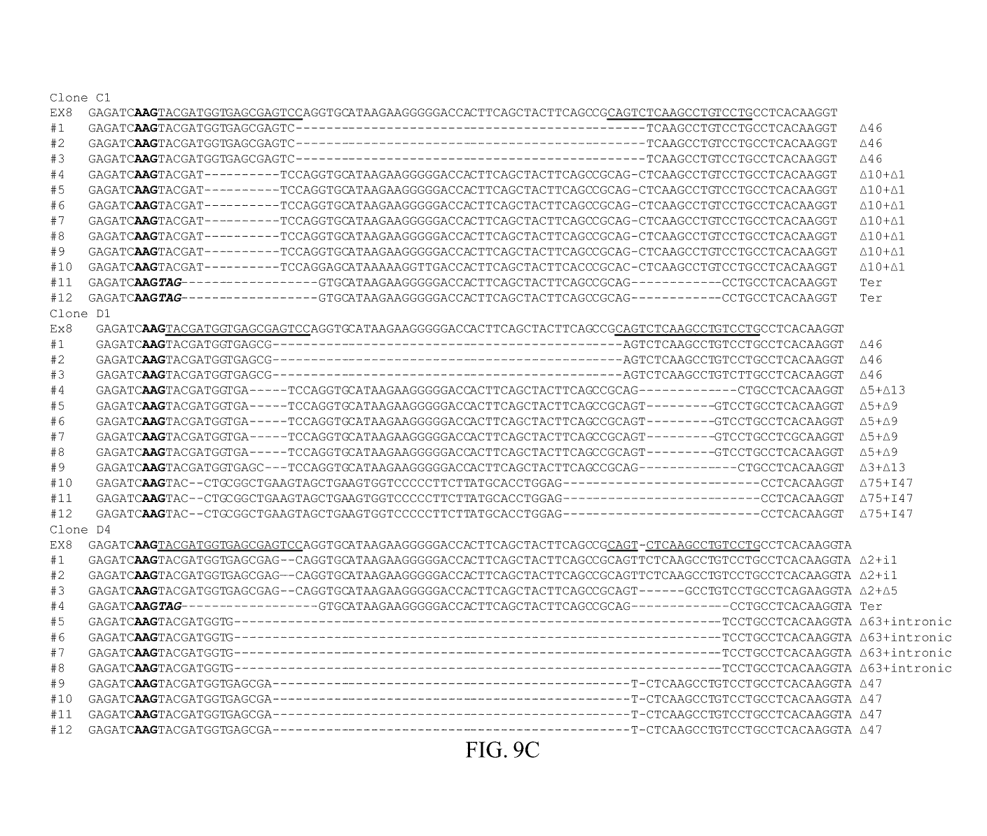

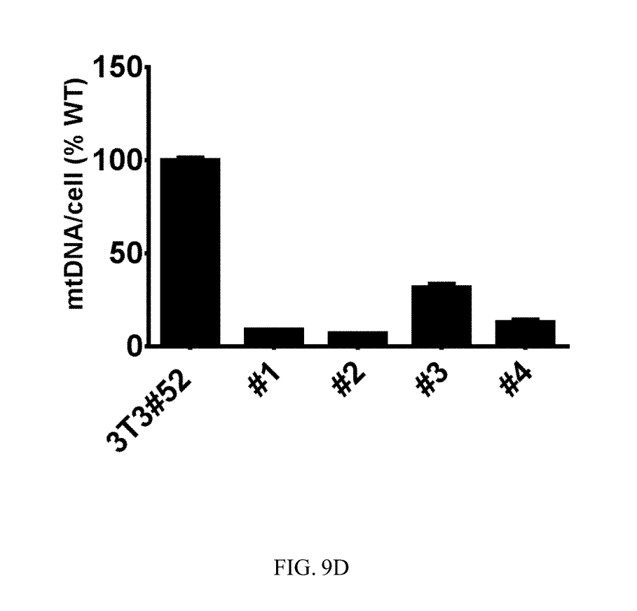

The procedure for the establishment of mouse cell lines with reduced mtDNA copy number described above relies on availability of cell lines with foxed Lig3 alleles, and therefore is difficult to generalize. To develop a general method, designed four single guide RNAs (sgRNAs) for CRISPR-Cas9 mediated inactivation of the Lig3 gene in mouse cells by introducing deletions in exon 1 or in exon 8 of this gene (two sgRNAs for each exon, FIG. 7H and Table 1). The method was validated by inactivating Lig3 in 3T3#52 cells expressing LigA as described in the Materials and Methods. Initial screening of the clones with putative inactivation of the Lig3 revealed substantial heterogeneity in mtDNA copy number in clones in which exon 1 was targeted (FIG. 6A). Interestingly, clones which retained Lig3 expression (possibly due to incomplete inactivation of all Lig3 alleles) had high mtDNA content (FIGS. 6C, 6D), whereas all tested clones with low mtDNA copy number had all Lig3 alleles inactivated (FIG. 9B). Of note, clone B6 (FIG. 6C), which had high mtDNA content, but no Lig3 detectable by western blotting, had one apparently active allele with two in-frame deletions (FIG. 9A). High mtDNA copy number in this clone is consistent with our previous observation that trace amounts of Lig3 are sufficient for the maintenance of normal mtDNA copy number (22). Remarkably, all tested clones in which exon 8 was targeted had reduced mtDNA copy number (FIGS. 6B, 6D and 9C). This observation may reflect the fact that exon 8 contains the active site of the enzyme, and therefore both in-frame and out-of-frame deletions in this exon are likely to be detrimental. The reduction in mtDNA copy number in 3T3#52 cells induced by substitution of the Lig3 with LigA was stable (FIG. 9D), which underscores the utility of the approach.

FIGS. 6A through 6D show a method for establishing mouse cell lines with reduced mtDNA copy number. FIGS. 6A and 6B show initial screening for mtDNA copy number in 3T3#53 clones expressing LigA, in which Lig3 exon 1 or exon 8 was targeted with CRISPR-Cas9. FIGS. 6C and 6D show the relationship between Lig3 inactivation and mtDNA copy number in clones with targeted exon 1 and exon 8.

mtDNA Depletion with EtBr is Accelerated in Cell Lines with Reduced mtDNA Copy Number

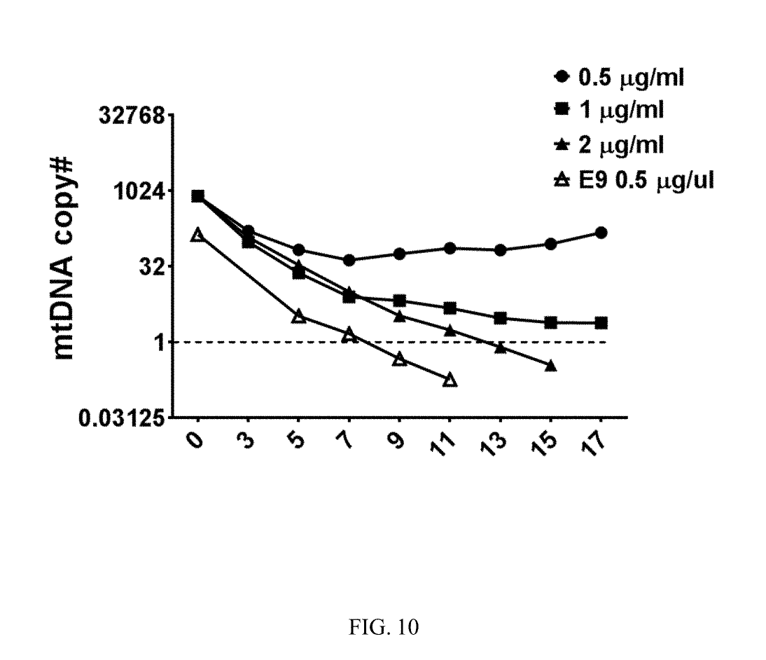

Applicants (21) and others (37) have shown that intracellular mtDNA cloning using partial depletion with EtBr is an effective means of inducing shifts in heteroplasmy (relative abundance of mtDNA haplotypes). However, prolonged treatment with EtBr represents rate-limiting step of this procedure. Next, whether kinetics of mtDNA depletion in response to EtBr treatment is altered in cells with reduced mtDNA copy number, which is mediated by substitution of the LigA for Lig3, was examined. Indeed, in both clones tested mtDNA depletion to the critical threshold of one copy per cell was achieved faster than in parental 3T3#52 cells (7 and 9 days vs. 12 days, respectively). Importantly, mtDNA depletion in cells depending on LigA for mtDNA maintenance was achieved with a lower concentration of EtBr to which parental cells were insensitive (FIG. 10).

Here, Applicants provide initial evidence for the existence of non-canonical low-capacity pathway for protein import into mitochondria, which facilitates mitochondrial uptake of some proteins lacking mitochondrial presequences, such as E. coli LigA protein and Chlorella virus ligase. Applicants observed that LigA targeted for import through canonical presequence-dependent pathway maintain normal mtDNA levels, whereas forcing cells to maintain their mtDNA by means of LigA import through this low-capacity pathway resulted in reduced mtDNA copy number. Together with Applicants' observation that 100-fold reduced mitochondrial levels of Lig3 were sufficient to maintain normal mtDNA copy number (22), this observation suggests low amount of LigA delivered to mitochondrial matrix through this pathway and therefore inefficiency of this pathway compared to presequence-driven import. Alternatively, this observation may be explained by intrinsic inefficiency of LigA compared to Lig3. However, this latter possibility is ruled out by observations that:
1. transduction of cells with low mtDNA copy number due to reliance on presequence-independent mitochondrial import of LigA with LigA fused to MTS restored mtDNA copy number. In these cells, LigA was imported through both presequence-dependent and -independent pathways; and
2. deletion of the MTS in the resulting cells lead to a drop in mtDNA copy number, ruling out a simple gene dosage effect of LigA fused to MTS.

Applicants further describe a means for harnessing this novel pathway by demonstrating that transduction of the target cells with a retrovirus encoding LigA devoid of MTS followed by inactivation of endogenous Lig3 by means of CRISPR-Cas9 RNA-guided nuclease resulted in the establishment of cell lines with reduced mtDNA copy number. This approach, therefore, can be used as a tool for studying mtDNA copy-number-dependent processes.

Cell lines, generated by this approach, also possess reduced growth rate and reduced OCR. They can be restored to WT status by transduction with viruses encoding DNA ligase fused to an MTS. Catalytically inactive mLig3 can partially rescue growth rates and maximal (uncoupled) respiration in these cells suggesting possible mtDNA maintenance-independent roles for Lig3 in cell cycle regulation and ETC function.

In this study, Applicants observed increased mtDNA copy number in response to different manipulations. Only one of the 4B6 clones in which mtDNA replication is supported by LigA without MTS had increased mtDNA copy number. Some 4B6 and 3T3 clones expressing both Lig3 and LigA had elevated mtDNA copy number. The magnitude of this increase, however, was much smaller than the magnitude of the drop in mtDNA copy number observed in clones with decreased mtDNA copy number.

### REFERENCES

1. Blackstone N W. The impact of mitochondrial endosymbiosis on the evolution of calcium signaling. Cell Calcium. 2015 March; 57(3):133-9.
2. Elkholi R, et al. Putting the pieces together: How is the mitochondrial pathway of apoptosis regulated in cancer and chemotherapy? Cancer Metab. 2014; 2:16.
3. Zorov D B, et al. Mitochondrial reactive oxygen species (ROS) and ROS-induced ROS release. Physiol Rev. 2014 July; 94(3):909-50.
4. Shah D I, et al. Mitochondrial Atpifl regulates haem synthesis in developing erythroblasts. Nature. 2012 Nov. 22; 491(7425):608-12.
5. Lill R, et al. The role of mitochondria in cytosolic-nuclear iron-sulfur protein biogenesis and in cellular iron regulation. Curr Opin Microbiol. 2014 Oct. 22; 22C:111-9.
6. Cha M Y, et al. The role of mitochondrial DNA mutation on neurodegenerative diseases. Exp Mal Med. 2015; 47:e150.
7. Wallace D C. Mitochondria and cancer. Nat Rev Cancer. 2012 October; 12(10): 685-98.
8. Li H Z, et al. A review of maternally inherited diabetes and deafness. Frontiers in bioscience (Landmark edition). 2014; 19:777-82.
9. Bratic A, Larsson N G. The role of mitochondria in aging. J Clin Invest. 2013 Mar. 1; 123(3):9517.
10. El-Hattab A W, Scaglia F. Mitochondrial DNA depletion syndromes: review and updates of genetic basis, manifestations, and therapeutic options. Neurotherapeutics. 2013 April; 10(2): 186-98.
11. Clay Montier L L, et al. Number matters: control of mammalian mitochondrial DNA copy number. J Genet Genomics. 2009 March; 36(3):125-31.
12. Nogueira C, et al. Syndromes associated with mitochondrial DNA depletion. Ital J Pediatr. 2014; 40(1):34.
13. Shokolenko I, et al. Oxidative stress induces degradation of mitochondrial DNA. Nucleic Acids Res. 2009 May; 37(8):2539-48.
14. Chen H, et al. Reduction and restoration of mitochondrial DNA content after focal cerebral ischemia/reperfusion. Stroke. 2001 October; 32(10):2382-7.
15. Mansouri A, et al. An alcoholic binge causes massive degradation of hepatic mitochondrial DNA in mice. Gastroenterology. 1999 July; 117(1):181-90.
16. Hsu C C, et al. Mitochondrial DNA alterations and mitochondrial dysfunction in the progression of hepatocellular carcinoma. World J Gastroenterol. 2013 Dec. 21; 19(47):8880-6.
17. Lee H C, et al. Somatic alterations in mitochondrial DNA and mitochondrial dysfunction in gastric cancer progression. World J Gastroenterol. 2014 Apr. 14; 20(14):3950-9.
18. Pastukh V, et al. Mutations in the passenger polypeptide can affect its partitioning between mitochondria and cytoplasm: Mutations can impair the mitochondrial import of DsRed. Mol Biol Rep. 2008 June; 35(2):215-23.
19. Giordano C, et al. Efficient mitochondrial biogenesis drives incomplete penetrance in Leber's hereditary optic neuropathy. Brain. 2014 February; 137(Pt 2):335-53.
20. Bianco A, et al. Mitochondrial DNA copy number differentiates the Leber's hereditary optic neuropathy affected individuals from the unaffected mutation carriers. Brain. 2015 Jul. 23.
21. Fayzulin R Z, et al. A method for mutagenesis of mouse mtDNA and a resource of mouse mtDNA mutations for modeling human pathological conditions. Nucleic Acids Res. 2015 May 19; 43(9):e62.
22. Shokolenko I N, et al. Mitochondrial DNA ligase is dispensable for the viability of cultured cells but essential for mtDNA maintenance. J Biol Chem. 2013 Sep. 13; 288(37):26594-605.
23. Sambrook J, Russel O W. Molecular Cloning. A laboratory manual. New York: Cold Spring Harbor Laboratory Press; 2001.
24. Zufferey R, et al. Multiply attenuated lentiviral vector achieves efficient gene delivery in vivo. Nat Biotechnol. 1997 September; 15(9):871-5.
25. Yu C, et al. A PCR Based Protocol for Detecting Indel Mutations Induced by TALENs and CRISPR/Cas9 in Zebrafish. PLoS One. 2014; 9(6):e98282.
26. van Leeuwen N, et al. Low mitochondrial DNA content associates with familial longevity: the Leiden Longevity Study. Age. 2014 June; 36(3):9629.
27. Mengel-From J, et al. Mitochondrial DNA copy number in peripheral blood cells declines with age and is associated with general health among elderly. Hum Genet. 2014 September; 133(9):1149-59.
28. Wang D, et al. Mitochondrial DNA copy number, but not haplogroup, confers a genetic susceptibility to leprosy in Han Chinese from Southwest China. PLoS One. 2012; 7(6):e38848.
29. Stringer H A, et al. Decreased skeletal muscle mitochondrial DNA in patients with statin-induced myopathy. J Neurol Sci. 2013 Feb. 15; 325(1-2):142-7.
30. Nakano Y, et al. Fatal case of mitochondrial DNA depletion with severe asphyxia in a newborn. Pediatr Int. 2011 April; 53(2):240-2.
31. Osterwalder M, et al. Dual RMCE for efficient re-engineering of mouse mutant alleles. Nat Methods. 2010 November; 7(11):893-5.
32. Shaner N C, et al. Improved monomeric red, orange and yellow fluorescent proteins derived from Discosoma sp, red fluorescent protein. Nat Biotechnol. 2004 December; 22(12): 1567-72.
33. Barazzoni R, et al. Effects of aging on mitochondrial DNA copy number and cytochrome c oxidase gene expression in rat skeletal muscle, liver, and heart. J Biol Chem. 2000 Feb. 4; 275(5):3343-7.
34. Barthelemy C, et al. Late-onset mitochondrial DNA depletion: DNA copy number, multiple deletions, and compensation. Ann Neural. 2001 May; 49(5); 607-17.
35. Olivera B M, Lehman I R. Diphosphopyridine nucleotide: a cofactor for the polynucleotide joining enzyme from Escherichia coli. Proc Natl Acad Sci USA. 1967 June; 57(6): 1700-4.
36. Ho C K, et al. Characterization of an ATP-dependent DNA ligase encoded by Chlorella virus PBCV-1. J Virol. 1997 March; 71(3):1931-7.
37. Acin-Perez R, et al. Respiratory complex III is required to maintain complex I in mammalian mitochondria. Mol Cell. 2004 Mar. 26; 13(6):805-15.

Now, therefore, the following is claimed:

1. A method for modulating mitochondrial DNA copy number in a mammalian cell, comprising the step of introducing at least one protein involved in mitochondrial DNA replication to the mammalian cell.

2. The method of claim 1, wherein the mitochondrial DNA copy number is increased by modulation.

3. The method of claim 2, wherein the mitochondrial DNA copy number is greater than the clinically normal average.

4. The method of claim 1, wherein each protein involved in mitochondrial DNA replication has at least one mitochondrial targeting sequence.

5. The method of claim 1, wherein each protein involved in mitochondrial DNA replication lacks mitochondrial targeting sequence.

6. The method of claim 1, wherein the mitochondrial DNA copy number is decreased by modulation, further comprising the steps of:
(a) selecting for the mammalian cell expressing protein involved in mitochondrial DNA replication; and
(b) selectively inactivating the protein in the protein-expressing mammalian cell.

7. The method of claim 6, wherein the mitochondrial DNA copy number is less than the clinically normal average.

8. The method of claim 1, wherein the protein involved in mitochondrial DNA replication is DNA ligase.

9. The method of claim 8, wherein the DNA ligase is Escherichia coli LigA.

10. The method of claim 1, wherein introduction of protein involved in mitochondrial DNA replication is characterized by expression of the protein.

11. The method of claim 1, wherein the protein involved in mitochondrial DNA replication is selected from bacteria or virus.

12. A mammalian cell clone that expresses a modulated mitochondrial DNA copy number, obtainable by a method comprising the step of introducing at least one protein involved in mitochondrial DNA replication to the mammalian cell clone.

13. The mammalian cell clone of claim 12, wherein the mitochondrial DNA copy number is increased.

14. The mammalian cell clone of claim 13, wherein the mitochondrial DNA copy number is greater than the clinically normal average.

15. The mammalian cell clone of claim 12, wherein each protein involved in mitochondrial DNA replication has at least one mitochondrial targeting sequence.

16. The mammalian cell clone of claim 12, wherein each protein involved in mitochondrial DNA replication lacks mitochondrial targeting sequence.

17. The mammalian cell clone of claim 12, wherein the mitochondrial DNA copy number is decreased, further comprising the steps of:
(a) selecting for the mammalian cell expressing protein involved in mitochondrial DNA replication; and
(b) selectively inactivating the protein in said protein-expressing cell.

18. The mammalian cell clone of claim 17, wherein the mitochondrial DNA copy number is less than the clinically normal average.

19. The mammalian cell clone of claim 12, wherein introduction of protein involved in mitochondrial DNA replication is characterized by expression of the protein.

20. The mammalian cell clone of claim 12, wherein the protein involved in mitochondrial DNA replication is selected from bacteria or virus.

21. The mammalian cell clone of claim 12, wherein the protein involved in mitochondrial DNA replication is DNA ligase.

22. The mammalian cell clone of claim 21, wherein the DNA ligase is Escherichia coli LigA.

23. A cell culture comprising a majority of cell clones with modulated mitochondrial DNA copy number.

24. The cell culture of claim 23, wherein the mitochondrial DNA copy number is greater than the clinically normal average.

25. The cell culture of claim 24, wherein the mitochondrial DNA copy number is less than the clinically normal average.
